(19) United States
(12) Patent Application Publication
(10) Pub. No.: US 2025/0359781 A1
(43) Pub. Date: Nov. 27, 2025

(54) METHOD, APPARATUS AND SYSTEM FOR DETERMINING BIOMECHANICAL STABILITY OF TARGET IN PERFORMING SIT-TO-STAND MOVEMENT

(71) Applicant: NEC Corporation, Tokyo (JP)

(72) Inventors: Ni Ni SOE, Singapore (SG); Charles Chi Hin Choy, Singapore (SG)

(73) Assignee: NEC Corporation, Tokyo (JP)

(21) Appl. No.: 19/207,495

(22) Filed: May 14, 2025

(30) Foreign Application Priority Data
May 21, 2024 (SG) 10202401435Y

(51) Int. Cl. A61B 5/11

(52) U.S. Cl. CPC A61B 5/1116; A61B 5/1128

(57) ABSTRACT

The present disclosure provides a method, an apparatus and a system for determining a biomechanical stability of a target in performing a movement from a sitting position to a standing position comprising: identifying, from each of a plurality of images, (i) an area on a ground which the target is in contact with and (ii) a line of gravity of the target projecting from a center of gravity of the target and perpendicular to the ground, wherein the each of the plurality of images showing one of a series of movements of the target moving from the sitting position to the standing position; and calculating a degree of biomechanical stability of the target in performing the movement from the sitting position to the standing position based on the areas and the lines of gravity of the target identified from the plurality of images.

### INCORPORATION BY REFERENCE

This application is based upon and claims the benefit of priority from Singapore patent application No. 10202401435Y, filed on May 21, 2024, the disclosure of which is incorporated herein in its entirety by reference.

### TECHNICAL FIELD

The present disclosure relates broadly, but not exclusively, to a method, an apparatus and a system for determining a biomechanical stability of a target in performing a movement from a sitting position to a standing position.

### BACKGROUND ART

Sit-to-stand (STS) movement is a fundamental daily activity that plays a crucial role in maintaining functional independence and quality of life. It assesses lower body strength, biomechanical stability, balance, and fall risk in older adults. It involves a complex interplay of various body segments and joints, making it a focal point in biomechanical and physiotherapy research. Therapists instruct the patients (e.g., Hemiplegic patients due to stroke) to rise safely from sitting while maintaining balance and weight-bearing.

Biomechanical analysis of the STS movement of a target, i.e., biomechanical stability of a target in performing an STS movement is essential for understanding the mechanics of this activity, determining biomechanical stability of the target, identifying potential movement dysfunctions of the target, and guiding therapeutic interventions. Understanding dynamics functional activities in elderly and impaired patients helps identify and correct abnormalities, preventing falls.

The sit-to-stand transition involves a multifaceted blend of biomechanics, motor function, physiological processes, and psychological elements. Essential aspects include body alignment, load distribution, timing, balance, coordination, strategy for compensation, movement fluidity, strength deployment, and mental processing.

Conventional techniques predominantly analyzed only the lower body, and lack comprehensive assessments of whole-body dynamics, particularly the upper body's influence on the transition from sitting to standing. In addition, assessment methods for biomechanical stability including balance and weight bearing have included professional observation by trained physical therapists visually and the use of force plates to evaluate balance and stability. Furthermore, current techniques utilizes computer vision technique and video analytic from one field of view, thus cannot provide weight-bearing assessments during this sit-to-stand (STS) motion, i.e., the weight distribution between the left and right sides of the body, and lack of in-depth qualitative evaluation. The use of multiple sensors also may not fully capture the entirety of movement patterns, stability, and weight distribution.

Therefore, there is a need for a method, an apparatus and a system to address the above challenges to determine a biomechanical stability of a target in performing a movement from a sitting position to a standing position.

Furthermore, other desirable features and characteristics will become apparent from the subsequent detailed description and the appended claims, taken in conjunction with the accompanying drawings and this background of the disclosure.

### SUMMARY

In a first aspect, the present disclosure provides a method for determining a biomechanical stability of a target in performing a movement from a sitting position to a standing position comprising: identifying, from each of a plurality of images, (i) an area on a ground which the target is in contact with and (i) a line of gravity of the target projecting from a center of gravity of the target and perpendicular to the ground, wherein the each of the plurality of images showing one of a series of movements of the target moving from the sitting position to the standing position; and calculating a degree of biomechanical stability of the target in performing the movement from the sitting position to the standing position based on the areas and the lines of gravity of the target identified from the plurality of images.

In a second aspect, the present disclosure provides an apparatus for determining a biomechanical stability of a target in performing a movement from a sitting position to a standing position comprising: at least one processor; and at least one memory including computer program code; the at least one memory and the computer program code configured to, with at least one processor, cause the apparatus at least to: identify, from each of a plurality of images, (i) an area on a ground which the target is in contact with and (i) a line of gravity of the target projecting from a center of gravity of the target and perpendicular to the ground, wherein the each of the plurality of images showing one of a series of movements of the target moving from the sitting position to the standing position; and calculate a degree of biomechanical stability of the target in performing the movement from the sitting position to the standing position based on the areas and the lines of gravity of the target identified from the plurality of images.

In a third aspect, the present disclosure provides a system for determining a biomechanical stability of a target in performing a movement from a sitting position to a standing position, the system comprises the apparatus of according to the second aspect and an image capturing apparatus for capturing the plurality of images and detecting the series of movements of the target from the plurality of images.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF DRAWINGS

The accompanying Figures, where like reference numerals refer to identical or functionally similar elements throughout the separate views and which together with the detailed description below are incorporated in and form part of the specification, serve to illustrate various embodiments and to explain various principles and advantages in accordance with a present embodiment, by way of non-limiting example only.

Embodiments of the disclosure will be better understood and readily apparent to one of ordinary skill in the art from the following written description, by way of example only, and in conjunction with the drawings, in which:

FIG. 1 shows a schematic diagram illustrating four different phases in performing a sit-to-stand (STS) movement.

FIG. 2 shows a schematic diagram illustrating sitting positions of four different persons (targets) and their respective foot positions.

FIG. 3 shows a block diagram illustrating an apparatus for determining a biomechanical stability of a target in performing a movement from a sitting position to a standing position according to various embodiments of the present disclosure.

FIG. 4 shows a flow chart illustrating a method for determining a biomechanical stability of a target in performing a movement from a sitting position to a standing position according to various embodiments of the present disclosure.

FIG. 5 shows a block diagram illustrating a system for determining a biomechanical stability of a target in performing a movement from a sitting position to a standing position according to an embodiment of the present disclosure.

FIG. 6 shows a flow chart 600 illustrating a method, for example, carried by the system in FIG. 5, for determining a biomechanical stability of a target in performing a movement from a sitting position to a standing position according to an embodiment of the present disclosure.

FIG. 7 shows four exemplary images recorded by an image capturing from a frontal view of a target moving from a sitting position to a standing position according to an embodiment of the present disclosure.

FIG. 8 shows four exemplary images recorded by an image capturing from a side view of a target moving from a sitting position to a standing position according to an embodiment of the present disclosure.

FIG. 9A shows a schematic diagram illustrating various landmarks that can be detected from a video/image recorded from a frontal view of a target according to an embodiment of the present disclosure.

FIG. 9B shows a schematic diagram illustrating various landmarks that can be detected from a video/image recorded from a side view of a target according to an embodiment of the present disclosure.

FIG. 10 shows a flow chart illustrating a method for detecting a position of a body segment of a target according to an embodiment of the present disclosure.

FIG. 11 shows a flow chart illustrating a method for processing the landmarks for side view according to an embodiment of the present disclosure.

FIG. 12 shows a flow chart illustrating a process for detecting four phases of sit-to-stand movement according to an embodiment of the present disclosure.

FIG. 13 shows an exemplary result of detection of phases of sit-to-stand using range of motion (ROM) of different body joints from a side view video according to an embodiment of the present disclosure.

FIG. 14 shows a flow chart illustrating a process for calculating smoothness and variability of joints of a target in performing the STS movement according to an embodiment of the present disclosure.

FIG. 15A a graph illustrating exemplary results of smoothness of joint angular movement of a target in performing an STS movement according to an embodiment of the present disclosure.

FIG. 15B a graph illustrating exemplary results of variability of joint angular movement of a target in performing an STS movement according to an embodiment of the present disclosure.

FIG. 16 shows schematic diagrams illustrating relationship of base of support (BOS), center of gravity (COG) and line of gravity (LOG) of a target when the target is in a standing position according to an embodiment of the present disclosure.

FIG. 17A shows a schematic diagram illustrating BOS of a target detected from a frontal view image when the target is in a sitting position according to an embodiment of the present disclosure.

FIG. 17B shows a schematic diagram illustrating BOS of a target detected from a side view image when the target is in a sitting position according to an embodiment of the present disclosure.

FIG. 18 shows a schematic diagram illustrating a relationship of BOS, COG and LOG of a target when the target is in a sitting position according to an embodiment of the present disclosure.

FIGS. 19A and 19B show two schematic diagrams illustrating two different standing positions of a target from a frontal view according to an embodiment of the present disclosure.

FIGS. 19C and 19D show another two schematic diagrams illustrating two other standing positions of the target from a side view according to an embodiment of the present disclosure.

FIG. 20 shows two images among a plurality of images of a target captured from a side view during a sit-to-stand movement and the BOS and LOG positions detected from the images according to an embodiment of the present disclosure.

FIG. 21 shows a graph illustrating exemplary detected distance between LOG and the center of BOS of a target during the STS movement based on side view images according to an embodiment of the present disclosure.

FIG. 22 shows a flow chart illustrating a method for processing the landmarks for frontal view according to an embodiment of the present disclosure.

FIG. 23A shows an image among a plurality of images of a target captured from a frontal view during a sit-to-stand movement, the BOS and LOG positions detected from the image and two graphs illustrating results of the y-axis difference and shifting of midpoint of joints, according to an embodiment of the present disclosure.

FIG. 23B shows a graph illustrating exemplary detected distance between LOG and the center of BOS of a target during the STS movement based on frontal view images according to an embodiment of the present disclosure.

FIG. 24 shows a flow chart illustrating the process of identifying left or right asymmetry according to an embodiment of the present disclosure.

FIGS. 25A to 25C show three graphs illustrating the distances of shoulder joints, hip joints and knee joints to the LOGs of a target measured from frontal view images during an STS movement, respectively, according to an embodiment of the present disclosure.

FIG. 26A shows a bar chart illustrating exemplary asymmetry indices of different body joints for the STS movement derived from frontal STS images according to an embodiment of the present disclosure.

FIG. 26B shows another bar chart exemplary asymmetry indices of different body joints for each phase of the STS movement derived from frontal STS images according to an embodiment of the present disclosure.

FIG. 27 shows a flow chart illustrating a process for calculating smoothness between left and right joints, variability between left and right joints, speed (velocity) of joint movement and acceleration of joint movement a target in performing the STS movement according to an embodiment of the present disclosure.

FIG. 28A shows a bar graph illustrating exemplary results of smoothness of left and right joints a target in performing an STS movement according to an embodiment of the present disclosure.

FIG. 28B shows a bar graph illustrating exemplary results of variability of left and right joints a target in performing an STS movement according to an embodiment of the present disclosure.

FIG. 28C shows a bar graph illustrating exemplary results of speed of left and right joints a target in performing an STS movement according to an embodiment of the present disclosure.

FIG. 28D shows a bar graph illustrating exemplary results of acceleration of left and right joints a target in performing an STS movement according to an embodiment of the present disclosure.

FIG. 29 shows a flow chart illustrating a process of calculating an overall balance and stability index corresponding to degree of biomechanical stability of the target in performing the STS movement according to an embodiment of the present disclosure.

FIG. 30A shows a radar chart illustrating trajectories of balance and stability index detected from side and frontal view images according to an embodiment of the present disclosure.

FIG. 30B shows another radar chart illustrating the overall balance and stability index according to an embodiment of the present disclosure.

FIG. 31 shows a schematic diagram of an exemplary computing device suitable for use to execute the method in FIG. 4 and implement the apparatus in FIG. 3.

### EXAMPLE EMBODIMENT

Embodiments of the present disclosure will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “receiving”, “calculating”, “determining”, “updating”, “generating”, “initializing”, “outputting”, “receiving”, “retrieving”, “identifying”, “dispersing”, “authenticating” or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer or a server or a cloud computing infrastructure. The computer readable medium may also include a hard-wired medium such as exemplified in the Internet system, or wireless medium such as exemplified in the GSM mobile telephone system. The computer program when loaded and executed on such a computer effectively results in an apparatus that implements the steps of the preferred method.

Various embodiments of the present disclosure relate to a method, an apparatus and a system for determining a biomechanical stability of a target in performing a movement from a sitting position to a standing position.

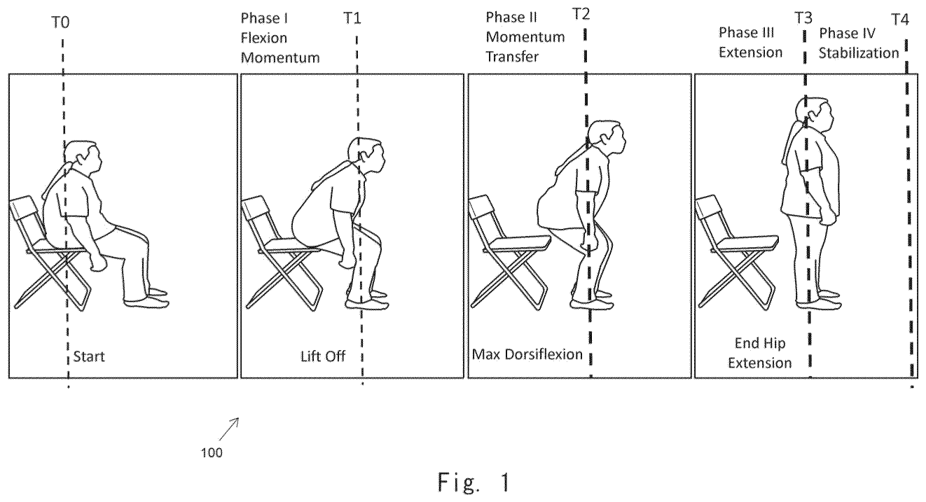

FIG. 1 shows a schematic diagram 100 illustrating four different phases in performing a sit-to-stand (STS) movement. An STS movement can be separated into four phases by five transitional (time) points (T0-T4). At T0, the target is at sitting position. Phase I is flexion-momentum phase which occurs between T0 and T1. In one example, the time point (or the frame number) at which a lift off of the hip of the target is detected is identified as T1. Phase II is momentum-transfer phase which occurs between T1 and T2. In this example, the time point (or the frame number) at which the maximum dorsiflexion is detected is identified as T2. Phase III is extension phase which occurs between T2 and T3. The time point (or the frame number) at which the end of hip extension is detected is identified as T3. Phase IV is stabilization phase which occurs between T3 and T4.

Currently, a sit-to-stand movement assessment is performed through manual clinical workflow for STS by a clinician using stopwatch. In such case, the range of motions is estimated through observation, the duration between phases and the number of repetition are measured manually and the quality of movement, the center of gravity movement and weight bearing asymmetry are observed. Such manual assessment may be useful clinically and provides helpful qualitative data, but it lacks precision and objectivity, very labor intensive for clinician, low reproducibility between different observers, limitations for research or detailed biomechanical analysis.

Stanford University Research published their research last year “Smartphone videos of the sit-to-stand test predict osteoarthritis and health outcomes in a nationwide study”. It is a self-guided quantitative motion analysis of the widely used five-repetition sit-to-stand test using a smartphone. Across 35 US states, 405 participants recorded a video performing the test in their homes. The video was taken for single view of 45 degrees angle from the front. They found that the quantitative movement parameters extracted from the smartphone videos were related to a diagnosis of osteoarthritis, physical and mental health, body mass index, age, and ethnicity and race. However, the quality of movement, center of gravity, and balance, and weight bearing were not captured.

Other existing solutions use force plates and motion capture system to perform sit-to-stand movement assessment. Motion capture systems are expensive and need dedicated facilities, many cameras and reflective markers and used in hospital and laboratories. Force plates alone cannot capture all the motion pattern and balance and weight bearing.

As mentioned earlier, a sit-to-stand transition involves a multifaceted blend of biomechanics, motor function, physiological processes, and psychological elements. Essential aspects include body alignment, load distribution, timing, balance, coordination, strategy for compensation, movement fluidity, strength deployment, and mental processing.

Conventional techniques predominantly analyzed only the lower body, and lack comprehensive assessments of whole-body dynamics, particularly the upper body's influence on the transition from sitting to standing. In addition, assessment methods for biomechanical stability including balance and weight bearing have included professional observation by trained physical therapists visually and the use of force plates to evaluate balance and stability. Furthermore, current techniques utilizes computer vision technique and video analytic from one field of view, thus cannot provide weight-bearing assessments during this sit-to-stand (STS) motion, i.e., the weight distribution between the left and right sides of the body, and lack of in-depth qualitative evaluation. The use of multiple sensors also may not fully capture the entirety of movement patterns, stability, and weight distribution.

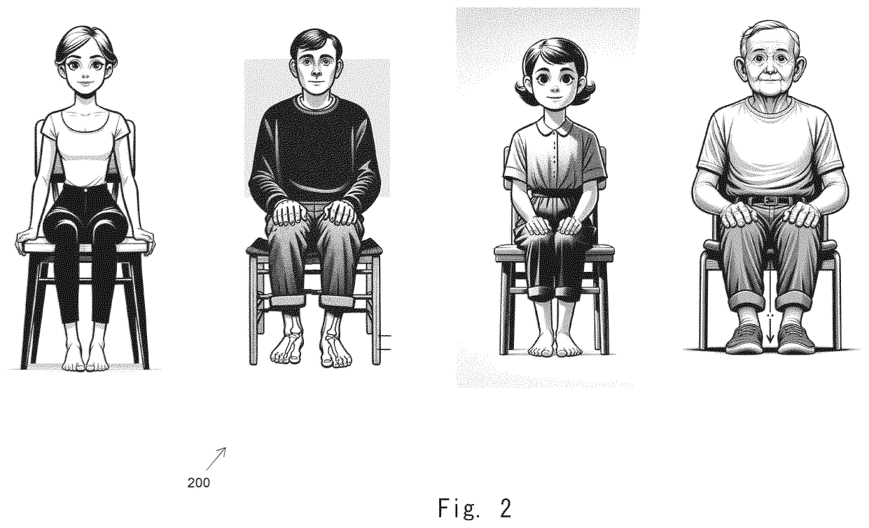

Besides, initial sitting position with different foot position is one of the important parameter to get balance and symmetric weight bearing during sit to stand. FIG. 2 shows a schematic diagram 200 illustrating sitting positions of four different persons (targets) and their respective foot positions.

Such parameter relating to the foot position of a target is not considered in the conventional techniques in assessment and determining the performance or stability of the target in performing a sit-to-stand movement. Patients need to be aware of their safety.

Therefore, there is a need for a method, an apparatus and a system to address the above challenges to determine a biomechanical stability of a target in performing a movement from a sitting position to a standing position so as to enhance objectivity and precision in healthcare, an automated system is crucial for conducting sit-to-stand evaluations, providing valuable biomechanical and physiotherapy insights in both clinical and home settings beyond subjective assessments.

Such method, apparatus and system may provide various advantages and values including: (i) the user is able to choose to record the sit-to-stand movement from both which provide more comprehensive biomechanical analysis; (ii) the transaction from initiating state through stabilizing upright posture are comprehensively assessed to provide insight into performance and strategy using dynamic range of motion (ROM) from the side view; (iii) various important factors captured from front view video are considered such as foot placement (e.g., toe in/toe out condition and the distance between feet), balance and stability measurement based on the distance between line of gravity and center of base, and weight bearing or asymmetry between left and right during STS movement; and (iv) relevant biomechanical factors in-depth analysis of the quality and control of sit-to-stand transitions are monitored.

The following terminologies will be used to describe in the present disclosure:
Center of mass (COM) or center of gravity (COG): Summation of center point of each body segment. The terms “COM” and “COG” may be used interchangeably.
Base of support (BOS): Area of object (including a target with other object in connection with the target) that is in contact with the ground
Center of BOS: Center point of the BOS.
Balance: The ability to maintain equilibrium or stability, or ability to control the center of mass over base of support (BOS). Range of motion (ROM): Extent or degree of movement that a joint or series of joints is capable of achieving in various directions.
Line of gravity (LOG): Vertical projection of center of mass, i.e., a line projecting from the COG and perpendicular to the ground.
Trajectory: a path followed by an object/a point or the pattern of movement observed during specific movement or any motion of a target. The trajectory can be detected by measuring and tracking the position of the point(s) such as joint or COG, or the body segment(s) in a video or images of the STS movement of the target.
Toe in and Toe out position: lateral rotational angle of the toe relative to the heel

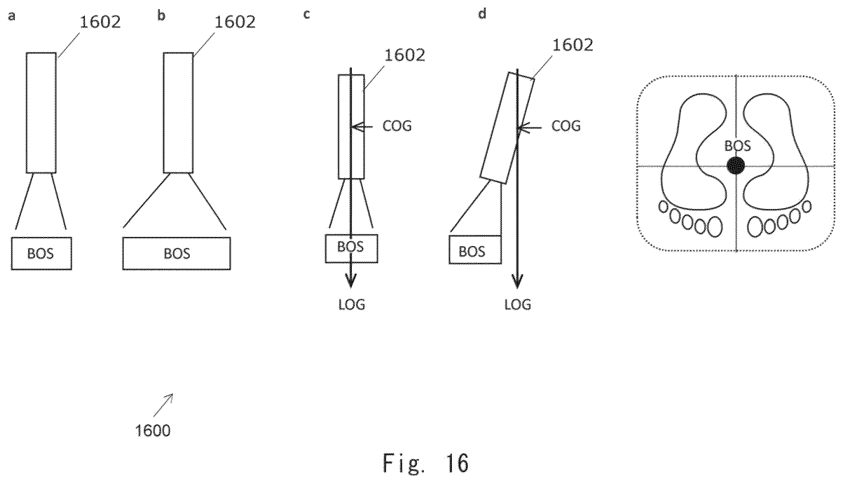

The terms may be further elaborated in FIGS. 16-21 and their accompanying description below.

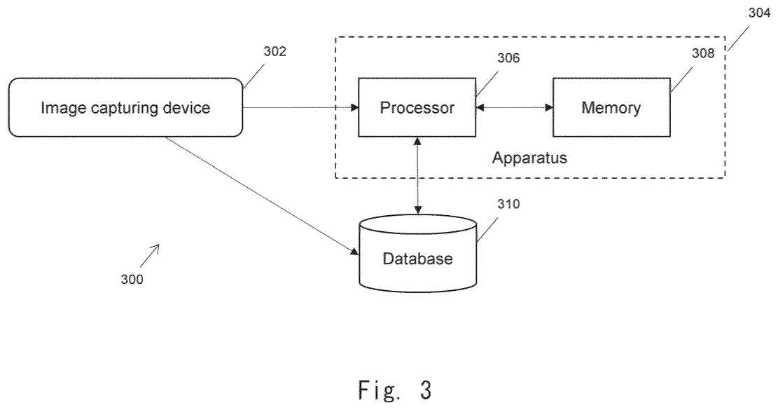

FIG. 3 shows a block diagram illustrating an apparatus 304 for determining a biomechanical stability of a target in performing a movement from a sitting position to a standing position according to various embodiments of the present disclosure.

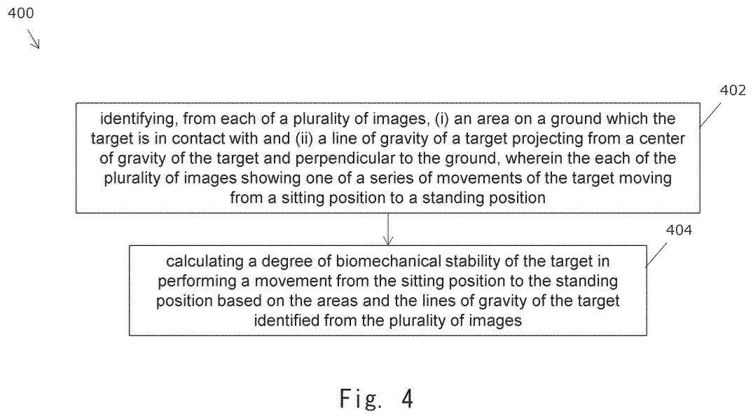

The managing of image or video input is performed by at least one image capturing device 302 and an apparatus 304. For the sake of simplicity, only one image capturing device 302 is illustrated. The system 300 comprises an image capturing device 302 in communication with the apparatus 304. In an implementation, the apparatus 304 may be generally described as a physical device comprising at least one processor 306 and at least one memory 308 including computer program code. The at least one memory 308 and the computer program code are configured to, with the at least one processor 306, cause the physical device to perform the operations described in FIG. 4. The processor 306 is configured to receive one or more images or videos from the image capturing device 302 or retrieve one or more images or videos from a database. Alternatively or additionally, the one or more images or videos captured by the image capturing device 302 is stored in a database 310, and the processor 306 is configured to retrieve the one or more images or videos from the database 310. It should be appreciated that the image capturing device 302 may be a part of the apparatus 304, forming a system 300 to perform operations described in FIG. 4.

The image capturing device 302 may be a device such as a mobile phone camera which provides a variety of data such as data relating to a facial and/or body feature and/or a movement of the facial and/or body feature of a person. In an implementation, appearance data derived from the image capturing device 302 may be stored in memory 308 of the apparatus 304 or a database 310 accessible by the apparatus 304. The data may include (i) facial and body feature data such as relative position, size, shape and/or contour of eyes, nose, cheekbones, jaw, chin, neck, shoulder, arm, and/or more particularly, jugular notch, shoulder center, glabella, sellion, chin, supramentale, sellion, pronasale and subnasale, and also iris pattern, skin colour, hair colour or a combination thereof, (ii) physical characteristic data such as height, body size, body ratio, shoulder width, distance between two facial and body features, length of limbs, hair colour, skin colour, apparels, belongings, equipment, other similar characteristics or combinations, and (iii) behavioral characteristic data such as movement, position of limbs, position of apparel/belonging/equipment, direction of movement, differential in movement direction, moving speed, frequency, movement patterns, the way or the time period a person or his/her facial and body feature stay stills or moves, other similar characteristics or combinations.

In an implementation, camera data such as location and resolution, and/or time data which includes a timestamp at which the person or his/her facial or body feature is identified may also be derived from the image capturing device 302. The camera data and/or time data may be stored in memory 308 of the apparatus 304 or a database 310 accessible by the apparatus 304 and the processor 306 is configured to identify and retrieve data, image or video based on the time data. It should be appreciated that the database 310 may be a part of the apparatus 304.

The apparatus 304 may be configured to communicate with the image capturing device 302 and the database 310. In an example, the apparatus 304 may receive, from the image capturing device 302, or retrieve from the database 310, one or more images or videos of a monitoring area corresponding to a field of view of the image capturing device 302, within which a frontal view and/or a side view of a person is detected.

FIG. 4 shows a flow chart 400 illustrating a method for determining a biomechanical stability of a target in performing a movement from a sitting position to a standing position according to various embodiments of the present disclosure. As shown in the exemplified method shown in FIG. 4, the apparatus 304 (or the processor 306 of the apparatus 304) may be configured to when in operation, is configured to perform the following steps:
step 402: identifying, from each of a plurality of images, (i) an area on a ground which the target is in contact with and (ii) a line of gravity of a target projecting from a center of gravity of the target and perpendicular to the ground, wherein the each of the plurality of images showing one of a series of movements of the target moving from a sitting position to a standing position; and
step 404: calculating a degree of biomechanical stability of the target in performing a movement from the sitting position to the standing position based on the areas and the lines of gravity of the target identified from the plurality of images.

It is noted that the plurality of images comprises a first plurality of images showing the series of movements of the target moving from the sitting position to the standing position from a side view and/or a second plurality of images showing the series of movements of the target moving from the sitting position to the standing position from a frontal view, and the step of calculating the degree of biomechanical stability comprises calculating a first degree of biomechanical stability of the target along a forward or backward direction and left or right direction based on the area and the lines of gravity of the target identified from the first and/or second plurality of images, respectively.

In one embodiment, in step 404, the memory 308 and the computer program code stored therein are configured to, with the processor 306 cause the apparatus 304 to further measure, from the each of the plurality of images, a shortest distance from the center of the area to a point on the line of gravity, wherein the degree of biomechanical stability is calculated based on the shortest distance. Additionally, the memory 308 and the computer program code stored therein are configured to, with the processor 306 may cause the apparatus 304 to further compare the shortest distance with a half of a length between two edges of the area on the ground, wherein the degree of biomechanical stability is calculated based on a result of the comparison.

In another embodiment, in step 402, the memory 308 and the computer program code stored therein are configured to, with the processor 306 cause the apparatus 304 to further identify, from the each of the plurality of images, a position of a center of gravity of each of body segments of the target at a pre-configured length, width and/or height of the each of the body segments; and determine the center of gravity of the target based on the position of the center of gravity of the body segment and a pre-configured weight percentage of the body segments making up a total weight of the target. Additionally or alternatively, the memory 308 and the computer program code stored therein are configured to, with the processor 306 cause the apparatus 304 to further detect, from the each of the plurality of images, positions of a toe, a heel and/or a foot of the target, wherein the area on the ground which the target is in contact with are identified based on the positions of the toe, the heel and/or the foot of the target.

In various embodiments of the present disclosure, there are four stages of movements performed by the target to complete the movement from the sitting position to the standing position. The memory 308 and the computer program code stored therein are configured to, with the processor 306 cause the apparatus 304 to further calculate, from the each of the plurality of images, one of (i) a first displacement of a position of a hip of the target along a direction parallel to the line of gravity, (ii) a second displacement of a position of a shoulder of the target along the direction parallel to the line of gravity, (iii) a first relative angle between two first body segments adjacent to the position of the hip; and (iv) a second relative angle between two second body segments adjacent to a position of a knee of the target; (v) a third relative angle between two third body segments adjacent to a position of an ankle of the target; and categorize the series of movements under one of four stages of movements performed by the target to complete the movement from the sitting position to the standing position based on the one of (i) the first displacement of the position of the hip, (ii) the second displacement of the position of the shoulder, (iii) the first relative angle between the two first body segments adjacent to the position of the hip, (iv) the second relative angle between the two second body segments adjacent to the position of the knee of the target and (v) the third relative angle between the two third body segments adjacent to the position of the ankle of the target.

In one embodiment, the memory 308 and the computer program code stored therein are configured to, with the processor 306 cause the apparatus 304 to further measure, from the each of the plurality of images, a relative angle formed between two fourth body segments adjacent to a position of a joint, wherein the joint is one of a hip, a knee, a hip, a trunk, a shoulder and an ear of the target; and calculate a change in the relative angles between the two fourth body segments around the position of joint measured across the plurality of images, wherein a smaller change or rate of the change in the relative angles indicates a smoothness of a movement of the joint in the series of movements. Additionally, the memory 308 and the computer program code stored therein are configured to, with the processor 306 cause the apparatus 304 to further (i) calculate a standard deviation of the relative angles between the two body segments around the position of the joint measured across the plurality of images, wherein a smaller standard deviation indicates a lower variability or greater stability of the movement of the joint in the series of movements and/or (ii) a speed and/or an acceleration of movements of the joint across the plurality of images; wherein the speed and/or the acceleration indicating an activity of the joint in the series of movements.

In another embodiment, the memory 308 and the computer program code stored therein are configured to, with the processor 306 cause the apparatus 304 to further measure, from the each of the plurality of images, shortest distances from positions of a left joint and a right joint of a joint to a point on the line of gravity, wherein the joint is one of a hip, a knee, an ankle, a trunk, a shoulder and an ear of the target; and calculate a degree of asymmetry between the left joint and the right joint in the series of movements based on the distances measured from the each of the plurality of images.

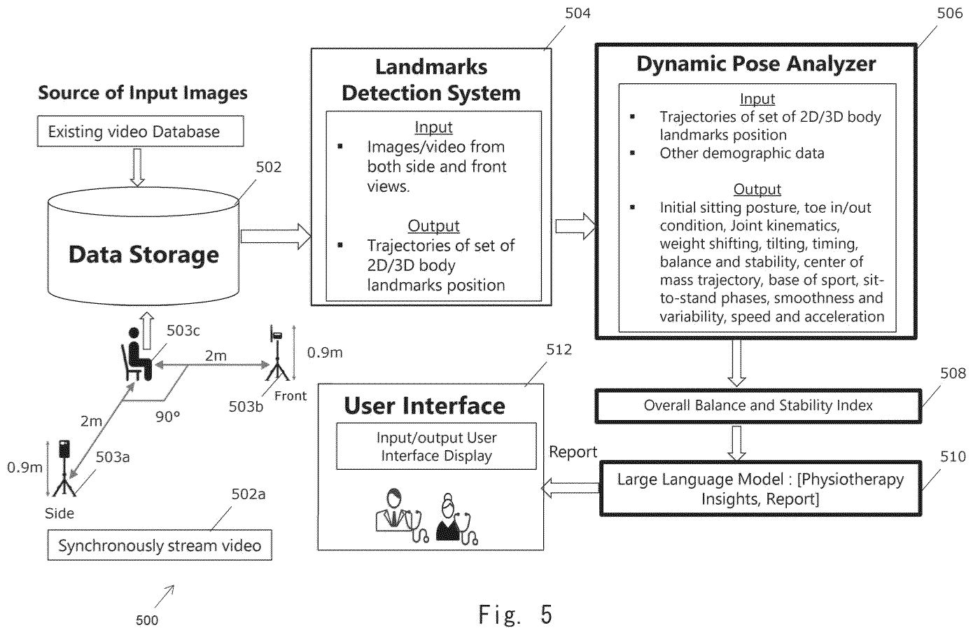

FIG. 5 shows a block diagram 500 illustrating a system for determining a biomechanical stability of a target in performing a movement from a sitting position to a standing position according to an embodiment of the present disclosure. The system may comprise a data storage 502, at least one image capturing devices 503a, 503b, a landmarks detection system (or unit) 504, a dynamic pose analyzer unit 506 and a processing unit 508 for calculating an overall balance and stability index corresponding to degree of biomechanical stability) and a large language model unit 510 for generating a physiotherapy insights and report to a user interface 512. The data storage may perform the same function as the database 310 illustrated in FIG. 3.

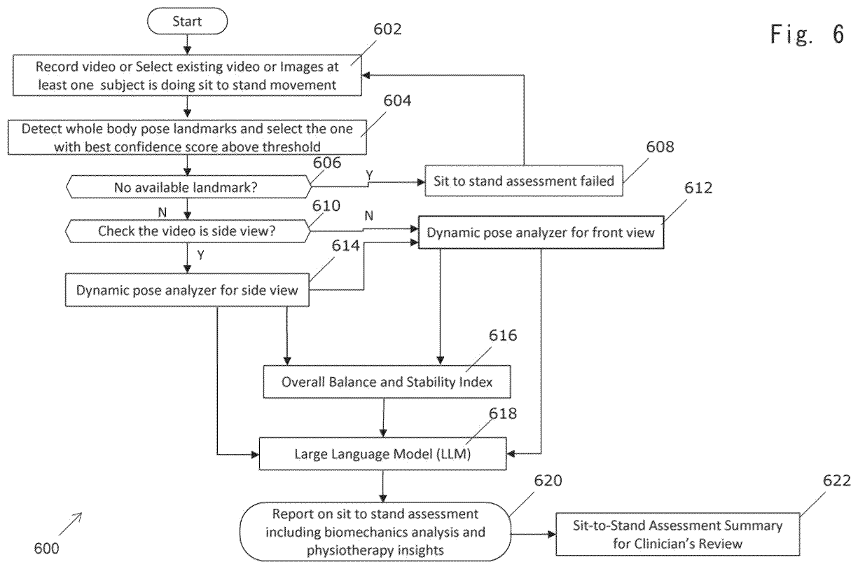

FIG. 6 shows a flow chart 600 illustrating a method, for example, carried by the system in FIG. 5, for determining a biomechanical stability of a target in performing a movement from a sitting position to a standing position according to an embodiment of the present disclosure.

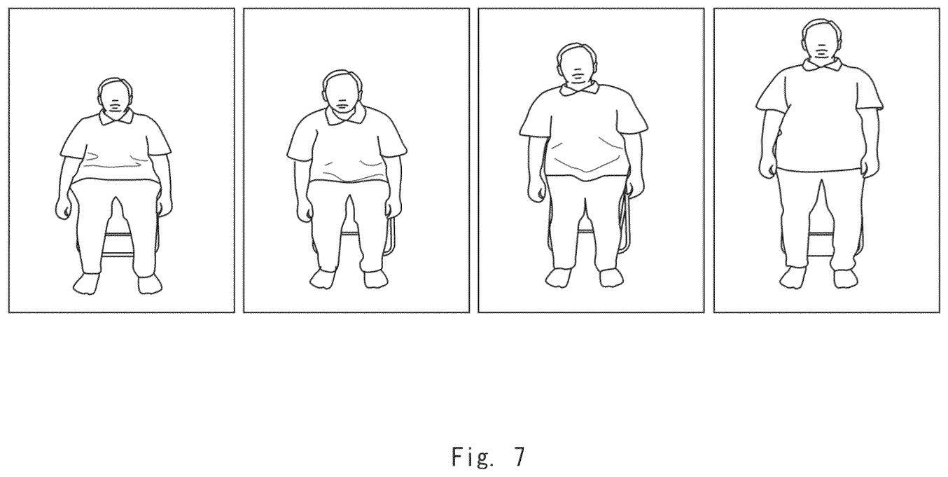

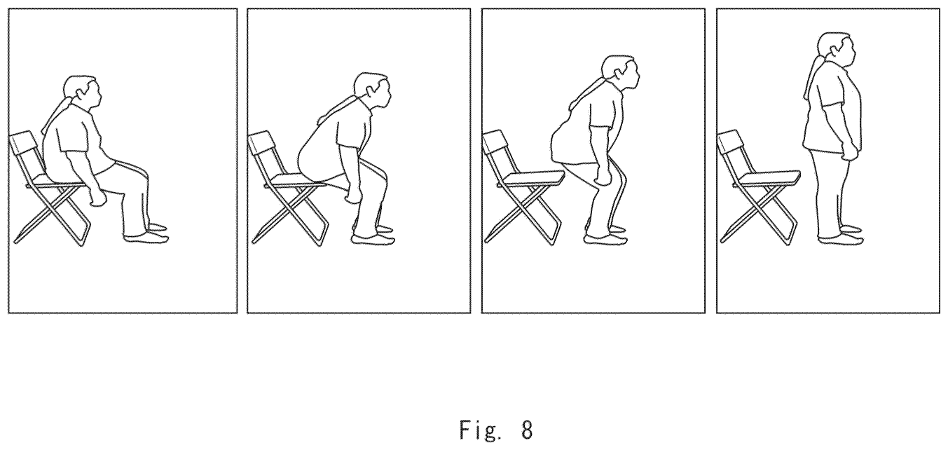

In step 602, synchronously stream video 502a of at least one subject performing a sit-to-stand movement may be recorded using two image capturing devices 503a, 503b from a frontal view and a side view of a target 503c simultaneously or selected from existing video or images in the data storage 502. FIG. 7 shows four exemplary images recorded by an image capturing from a frontal view of a target moving from a sitting position to a standing position according to an embodiment of the present disclosure. FIG. 8 shows four exemplary images recorded by an image capturing from a side view of a target moving from a sitting position to a standing position according to an embodiment of the present disclosure. In one alternative implementation, the video or images from the frontal view and the side view may be taken separately using one image capturing device. In such case, at least one view of video or image sequences of the target performing sit-to-stand movement may be sufficient to calculate a degree of biomechanical stability of the target in performing the movement.

The landmark detection system 504 may receive the images/video from both side and frontal views as an input and generate an output relating to a set of 2D/3D body landmark positions and their trajectories. In particular, in step 604, the landmark detection system 504 may detect a whole body pose landmarks and select the one with best confidence score above a threshold score may be carried by the landmark detection system. In step 606, the landmark detection system 504 may determine if there is no available landmark. If there is indeed no available landmark, step 608 is carried out where the sit-to-stand assessment failed; otherwise step 610 is carried out. In step 610, the landmark detection system 610 may check if the video is side view and if so, step 614 is carried out where the dynamic pose analyzer unit 506 will process the landmark(s) for side view; otherwise, step 612 is carried out where the dynamic pose analyzer unit 506 will process the landmark(s) for front view).

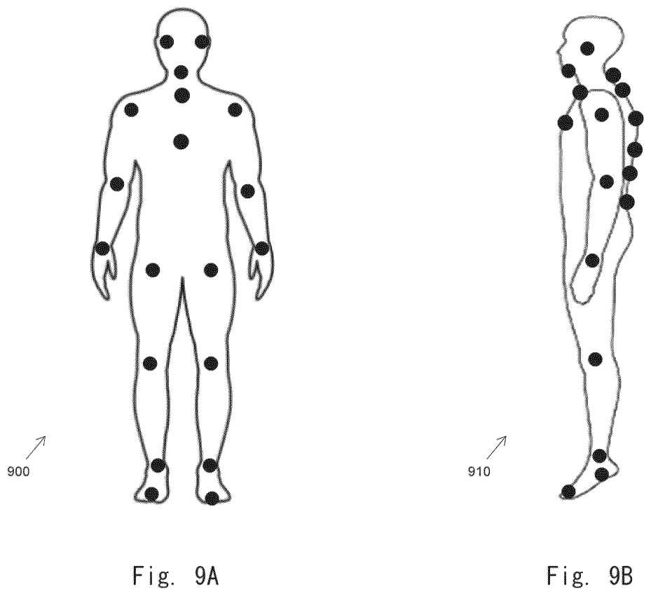

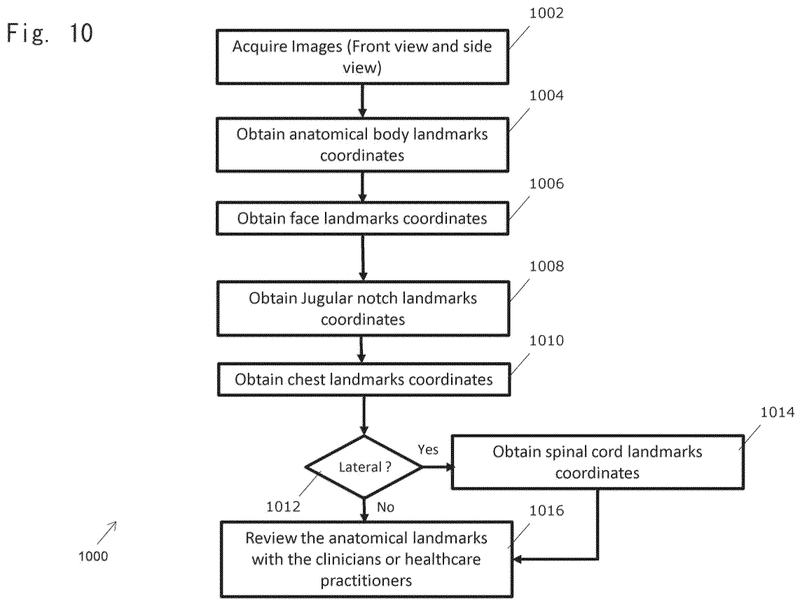

FIG. 9A shows a schematic diagram 900 illustrating various landmarks that can be detected from a video/image recorded from a frontal view of a target according to an embodiment of the present disclosure. FIG. 9B shows a schematic diagram 910 illustrating various landmarks that can be detected from a video/image recorded from a side view of a target according to an embodiment of the present disclosure. FIG. 10 shows a flow chart 1000 illustrating a method, for example, carried by the landmark detection system 504 in FIG. 5, for detecting a position of a body segment (or body landmark position) of a target according to an embodiment of the present disclosure. In step 1002, images are acquired from a frontal view and/or a side view of a target. In step 1004, a step of obtaining anatomical body landmarks coordinates (e.g., x-y coordinates) is carried out. In step 1006, a step of obtaining face landmarks coordinates is carried out. In step 1008, a step of obtaining jugular notch landmarks coordinates is carried out. In step 1010, a step of obtaining chest landmarks coordinates is then carried out. In step 1012, it is determined whether the image shows a lateral (side) view of the target, if so, step 1014 is carried out where spinal cord landmarks coordinates are obtained; otherwise step 1016 is carried out. In step 1016, the anatomical landmarks may be displayed and then reviewed with the clinicians or healthcare practitioners.

Returning to FIG. 5, the dynamic pose analyzer unit 506 may receive the output from the landmark detection system 504 and other demographic data to generate an output relating to initial sitting posture, toe in/out condition, Joint kinematics, weight shifting, tilting, timing, balance and stability, center of mass trajectory, base of sport, sit-to-stand phases, smoothness and variability, speed and acceleration.

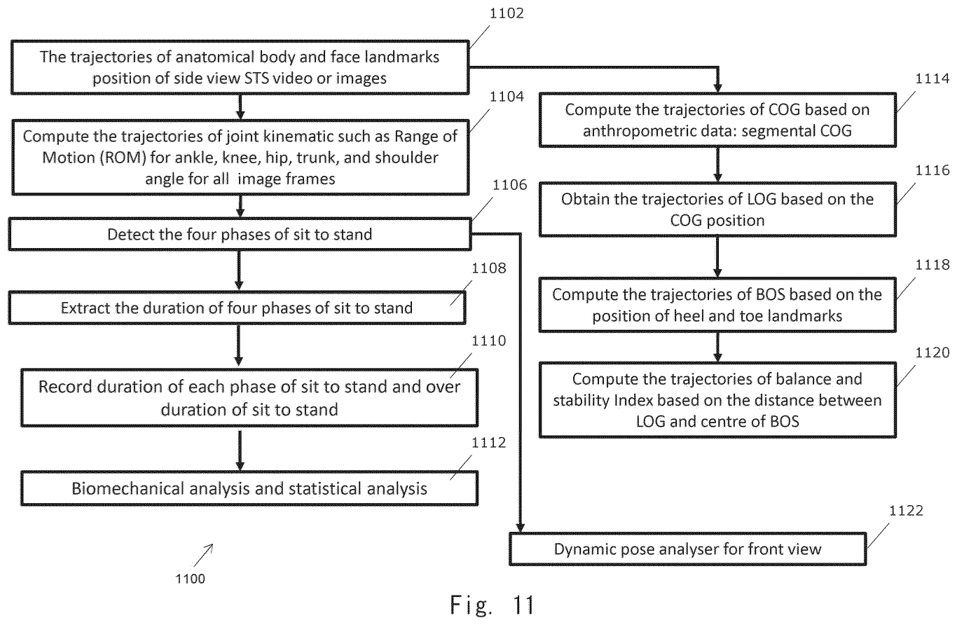

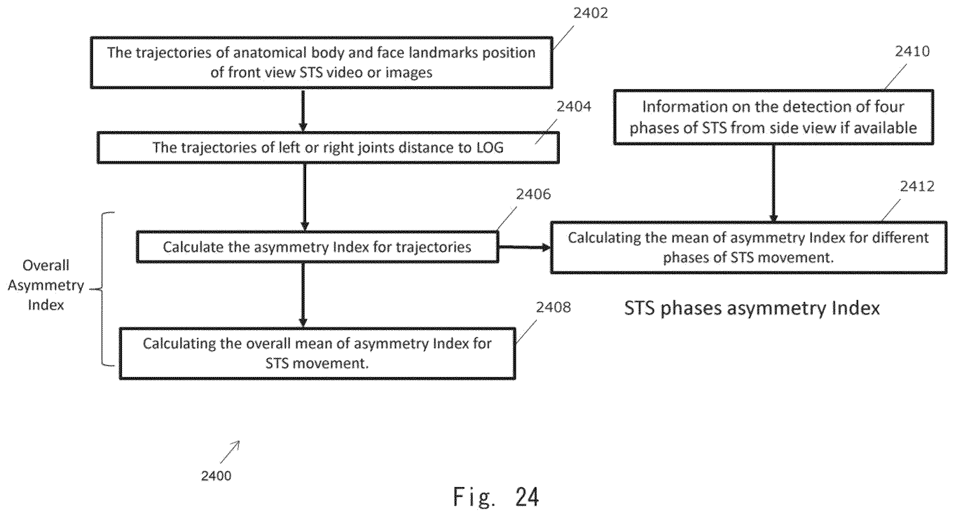

FIG. 11 shows a flow chart 1100 illustrating a method, for example, carried by the dynamic pose analyzer unit 506 in FIG. 5 for processing the landmarks for side view according to an embodiment of the present disclosure. In step 1102, the trajectories of anatomical body and face landmarks positions of video or images of a target performing an STS movement from a side view is identified. In step 1104, a step of computing the trajectories of joint kinematic such as range of motion (ROM) of ankle, knee, hip, trunk and shoulder angle for all image frames or every specific (three or two) image frame is carried out. In step 1106, a step of detecting four phases of sit-to-stand movement is carried out. The detected four phases will be further processed, with frontal view video or images of the target performing the STS movement in step 1122. More details on the processing of the landmarks for frontal view by the dynamic pose analyzer unit 506 will be shown in FIG. 24.

In step 1108, a step of extracting the duration of the four STS movement phases is carried out. In step 1110, a step of recording/calculating the duration of each phase of sit-to-stand and over duration of sit-to-stand is carried out. In step 1112, a step of generating a result of biomechanical analysis and statistical analysis (e.g., smoothness, variability) is carried out.

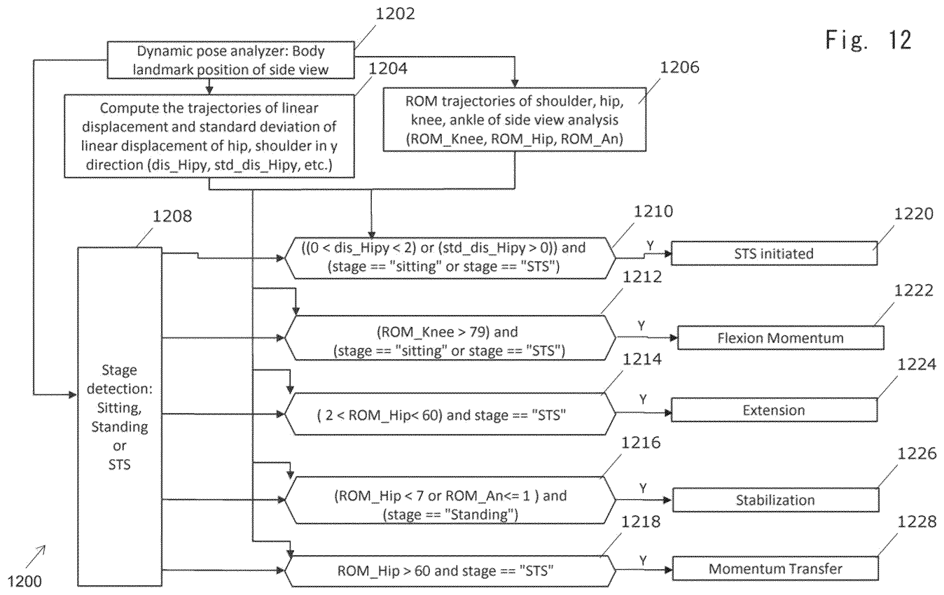

FIG. 12 shows a flow chart 1200 illustrating a process for detecting four phases of sit-to-stand movement according to an embodiment of the present disclosure. The process may start in step 1202 as the dynamic pose analyzer unit performs its intended function using body landmark positions identified from side view of a target performing the sit-to-stand movement. In step 1204, a step of computing the trajectories of linear displacement and standard division of linear displacement of hip, shoulder in y-direction (e.g., dis_Hipy, std_dis_Hipy, etc.) is carried out. In step 1206, a step of identifying the range of motion (ROM) trajectories of shoulder, hip, knee, ankle of side view analysis (ROM_knee, ROM_hip, ROM_Ankle) is carried out. In step 1208, stage detection is carried out. In this disclosure, there may be three major stages: (i) sitting, (ii) standing and (iii) during sit-to-stand movement (herein may refer to as “STS”). As shown in FIG. 1, an STS stage can be further divided and categorized into four phases separated by five transitional (time) points (TO-T4). At T0, STS is initiated. Phase I is flexion-momentum phase which occurs between T0 and T1. Phase II is momentum-transfer phase which occurs between T1 and T2. Phase III is extension phase which occurs between T2 and T3. Phase IV is stabilization phase which occurs between T3 and T4. The computed trajectories of linear displacement and standard deviation of linear displacement of hip, shoulder in y-direction and the ROM trajectories of shoulder, hip, knee and ankle of side view analysis computed in steps 1204, 1206 are used to identify the transitional points and thus categorize each phase of the STS movement.

For example, in step 1210, when it is determined (i) the linear displacement of hip is between 0 to 2 or standard division of linear displacement is larger than 0; and (ii) the stage is current sitting or STS stage, T0 or an STS initiated time point is identified. In step 1212, when it is determined that the ROM of knee joint is larger than 79 and the stage is either sitting or STS, T1 is identified and, with T0, a flexion momentum phase (phase 1) is categorized. In step 1214, when it is determined that the ROM of hip join is between 2 and 60 and the stage is STS stage, T3 is identified and, with T2, an extension phase (phase 3) can be categorized. In step 1216, when it is determined that (i) the ROM of hip joint is smaller than 7 or the ROM of ankle joint is smaller than or equal to 1, and (ii) the stage is standing stage, T4 is identified and, with T3, a stabilization phase (phase 4) can be categorized. In step 1218, when it is determined that the ROM of hip join is larger than 60 and the stage is STS, T2 is identified and, with T1, a momentum transfer phase (phase 2) can be categorized. It is noted that although a rule based model is illustrated for detection of stages and phases, such detection can be carried out through machine learning based model instead (not shown).

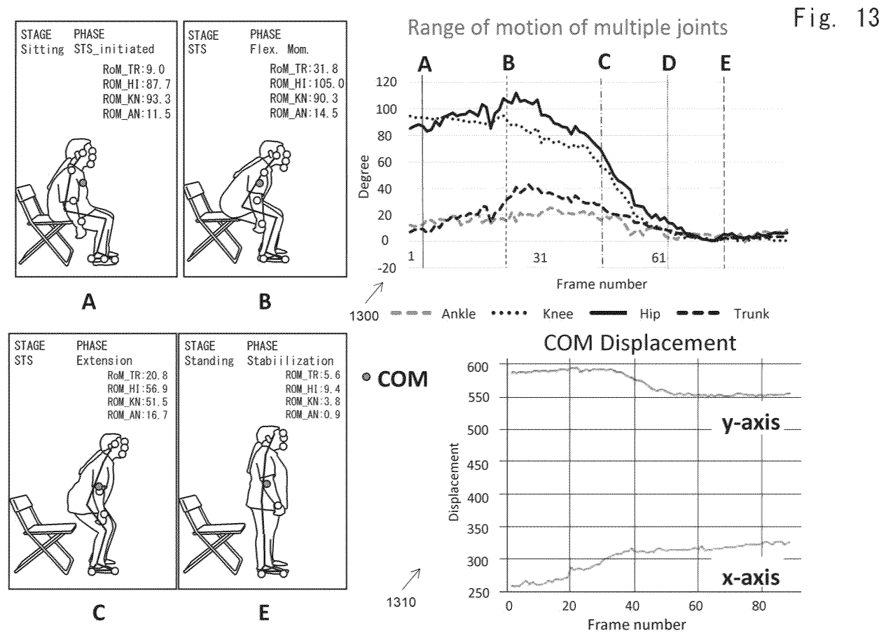

FIG. 13 shows an exemplary result of detection of phases of sit-to-stand using range of motion (ROM) of different body joints from a side view video according to an embodiment of the present disclosure. In this embodiment, body joints such as ear, shoulder, hip, trunk, ankle are identified, and the ROM of each joint (or the relative angle between two body segments around each joint) of the target to perform the STS movement is measured and tracked throughout the video. Additionally, center of mass (COM) of the target is also measured and tracked throughout the video, and the trajectories of the COM may also be used to identify the transitional (time) points of the phases.

Based on the trajectories of ROM of the joints (graph 1300 in FIG. 13) and optionally the COM (graph 1310 in FIG. 13) it is determined that an STS movement is initiated, and the start of flexion momentum phase is identified at point A (frame number 3, T0), the STS movement transitions to momentum transfer phase at point B (frame number 24, T1), to extension phase at point C (frame number 48, T2), to stabilization phase at point D (frame number 64, T3) and to the end of the stabilization phase at point E (frame number 73, T4). The durations for complete the full STS movement and each phase of the STS movement are measured and recorded based on the frames from which the transitional (time) points A-E (frame number 3, T0-frame number 73, T4) are detected (step 1110 in FIG. 11).

Table 1 shows a summary of the phase detection result shown in FIG. 13.

TABLE 1

| Phase | Start Frame | Stop Frame | Duration (s) |
|---|---|---|---|
| Flexion momentum | 3 | 23 | 0.667 |
| Momentum transfer | 24 | 48 | 0.8 |
| Extension | 48 | 64 | 0.533 |
| Stabilization | 64 | 73 | 0.3 |
| Total STS | 3 | 73 | 2.9 |

In addition, in step 1112, biomechanical analysis and statistical analysis relating to the joint may be carried out through other joint movement related parameters derived from the joints ROM trajectories detected from side view images. Such analysis is valuable for understanding joint behavior during movement and identifying areas that may require attention or further analysis, especially in contexts like physical therapy, sports science, or ergonomics.

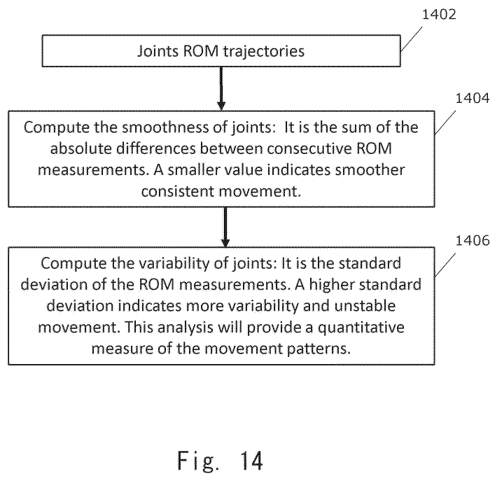

FIG. 14 shows a flow chart 1400 illustrating a process for calculating smoothness and variability of joints of a target in performing the STS movement according to an embodiment of the present disclosure. In step 1402, joints ROM trajectories are determined from a series of movement in performing an STS movement from a side view video or images (see graph 1310 in FIG. 13). In step 1404, a step of calculating the smoothness of joints is carried out. The smoothness of a joint relates to a sum of the absolute differences between consecutive ROM measurements from the series of movement in performing the STS movement from the video or images. It may be equivalent to an average rate of change of a joint in performing the STS movement and can be calculated using equation (1):

$$S = {\sum }_{i = 1}^{i = n - 1} \vert {ROM}_{i + 1} - {ROM}_{i} \vert \qquad equation ( 1 )$$

### Where

ROM=[ROM₁, ROM₂, . . . , ROMₙ] be the sequence of Range of Motion (ROM) measurements at a joint
n is the total number of ROM measurements
ROMᵢ is the i^{th} ROM measurement
   |ROMᵢ₊₁−ROMᵢ| is the absolute difference between consecutive ROM measurements.

A smaller value of S indicates that there are smaller changes between consecutive measurements, which implies a smoother movement. In step 1406, a step of calculating the variability of joints is carried out. It is the standard deviation of the ROM measurements. A higher standard deviation indicates more variability and unstable movement. This analysis will provide a quantitative measure of the movement patterns.

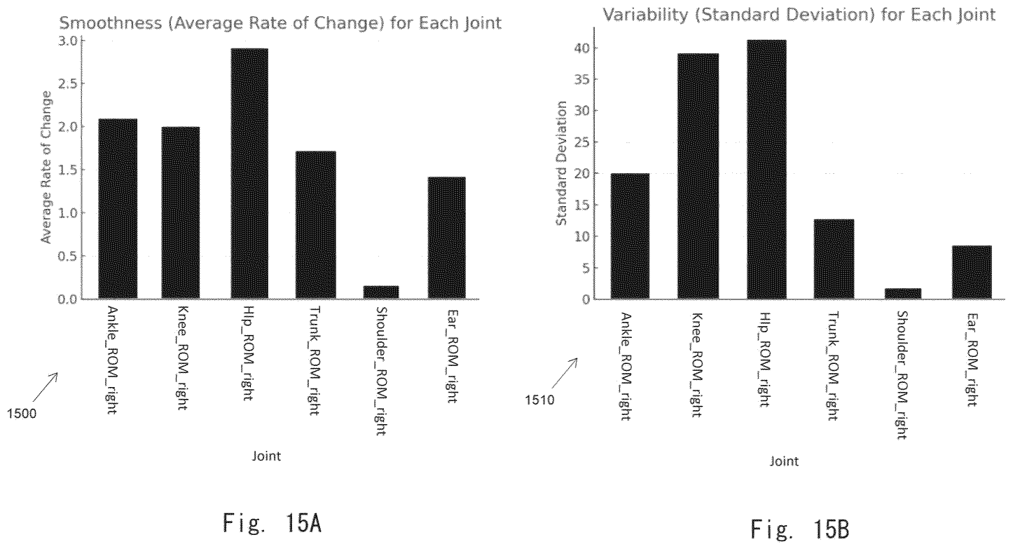

FIGS. 15A and 15B show two graphs 1500, 1510 illustrating exemplary results of smoothness and variability of joint angular movement of a target in performing an STS movement, taking the video from the right side, according to an embodiment of the present disclosure, respectively. The joints include right ankle joint, right knee joint, right knee joint, right trunk joint, right shoulder joint and right ear joint. From the results, the following insights can be derived:
the shoulder joint is the smoothest and least variable, indicating consistent and controlled movement;
the hip and knee joints display the highest variability and least smoothness, suggesting a broader range of motion and possibly less controlled or more dynamic movement; and
the ankle and ear joints exhibit moderate levels of smoothness and variability.

Returning to FIG. 11, in step 1114, the trajectories of anatomical body and face landmarks positions of video or images of the target performing an STS movement from a side view identified in step 1102 may be used for computing the trajectories of COG of various body segments of the target based on anthropometric data (segmental COG). In step 1116, a step of obtaining the trajectories of a line of gravity (LOG) is carried out based on the segmental COG positions. In step 1118, a step of computing the trajectories of base of support (BOS) is carried out based on the position of heel and toe landmarks. In step 1120, a step of computing the trajectories of balance and stability index based on the distances between LOG and center of BOS. Such output from the dynamic post analyzer unit 506 will be used by a processing unit 508 to calculate an overall balance and stability index of the target in performing the sit-to-stand movement.

To carry out step 1116, it is important to know the location of the effective center of gravity (or mass) of segments. It is noted that gravity actually pulls on every particle of mass, therefore giving each part weight. For the body, each body segment is treated as the smallest division of the body, thus a segmental COG for each individual body segment or group of body segments can be determined. In various embodiments, such segmental COGs are used to calculate and identify a position of a COG (COM) of the target. It is noted that the terms COM and COG can be used interchangeably in this present disclosure.

In one exemplary embodiment, from an image frame of a frontal view of the target, the COG of each body segment of the target is determined based on anthropometric data indicating a pre-configured segment length, segment width, segment height out of the total length, width, height of the body segment or a ratio thereof (e.g., percentage of segment length) at which the COG of each body segment resides.

For example, when the target is in sitting position, from the image of a right leg of the target from a side view, the COGs of the thigh segment (1), leg segment (2) and foot segment (3) are determined, for example, based on anthropometric data.

Table 2 shows the exemplary positions of the COGs of right thigh, leg and foot segments of the target determined from a side view of the target when the target is in sitting position as well as their weight percentages.

TABLE 2

| Segment | x (cm) | y (cm) | % W |
|---|---|---|---|
| 1, thigh | 17.3 | 51.3 | 10.6 |
| 2, leg | 42.5 | 32.8 | 4.6 |
| 3, foot | 45 | 3.3 | 1.7 |

In particular, the leg of is fixed at 90 degrees in the sitting position. Table 2 gives COGs and weights (as % of total body weight W) of segments 1, 2, and 3. Given the COGs of each segment of the lower body segment, the position/coordinate (x_{CG}, y_{CG}) of COG of the lower body segment can be determined. The following shows an example calculation of the x-coordinate of COG of the lower body segment using the sum of moments of each segment (thigh, leg and foot).

$${SM}_{O} = {x}_{CG} \{ {W}_{1} + {W}_{2} + {W}_{3} \} = {x}_{1} {W}_{1} + {x}_{2} {W}_{2} + {x}_{3} {W}_{3}$$

$${x}_{CG} = \{ {x}_{1} {W}_{1} + {x}_{2} {W}_{2} + {x}_{3} {W}_{3} \} / ( {W}_{1} + {W}_{2} + {W}_{3} ) = \{ 17.3 ( 0.106 W ) + 42.5 ( 0.046 W ) + 45 ( 0.017 W ) \} / ( 0.106 W + 0.046 W + 0.017 W )$$

$${x}_{CG} = 26.9 cm$$

Similarly, x-y coordinates of various joints (e.g., shoulder, elbow, hip, knee, ankle) of the target can be identified from an image of the target. Such positions of the joints can then be used to determine a combined COG of the target at this instance when the image was taken, for example, based on COGs of various body segments of the target and anthropometric data.

It is noted that stability is directional. An object or person can be more stable in one direction than another. Stability affect by not only the size of the base of support but the horizontal distance between the line of gravity and the edge of the base of support in the direction, and the high of center of the gravity. In many cases, LOG must remain within the BOS region to maintain equilibrium and stability.

According to various embodiments of the present disclosure, an area on a ground which the target is in contact (e.g., BOS) and a LOG of the target are identified and tracked across a video or images showing a target performing an STS movement from a sitting position to a standing position is, and a balance and stability index corresponding to a degree of biomechanical stability of a target in performing an STS movement from a sitting position to a standing position is calculated based on the identified areas and LOGs. In one implementation, the distance (d) between a center of BOS and the LOG (or the shortest distance between the the center of BOS to a point of the LOG) is measured in each instance/image and tracked across the video or images and such degree of biomechanical stability is calculated based on the distance (d)

FIG. 16 show schematic diagrams 1600 illustrating relationship of base of support (BOS), center of gravity (COG) and line of gravity (LOG) of a target when the target is in a standing position according to an embodiment of the present disclosure. In particular, the blocks 1602 in the diagrams 1600 represents an upper body segment of a target and the two lines extending from one side of the blocks represents two leg segments of the target when the target is in standing position. The sub-diagram (a) in FIG. 16 shows a smaller BOS when a target is standing normally while The sub-diagram (b) in FIG. 16 shows a larger BOS when the target is standing feet apart. The sub-diagram (c) in FIG. 16 shows exemplary BOS, LOG and position of COG of the target. The LOG lies within the BOS when the body of the target is stable. The sub-diagram (d) in FIG. 16 shows another exemplary BOS, LOG and position of COG of the target. In this case, the body of the target is not stable as the LOG lies outside of the BOS.

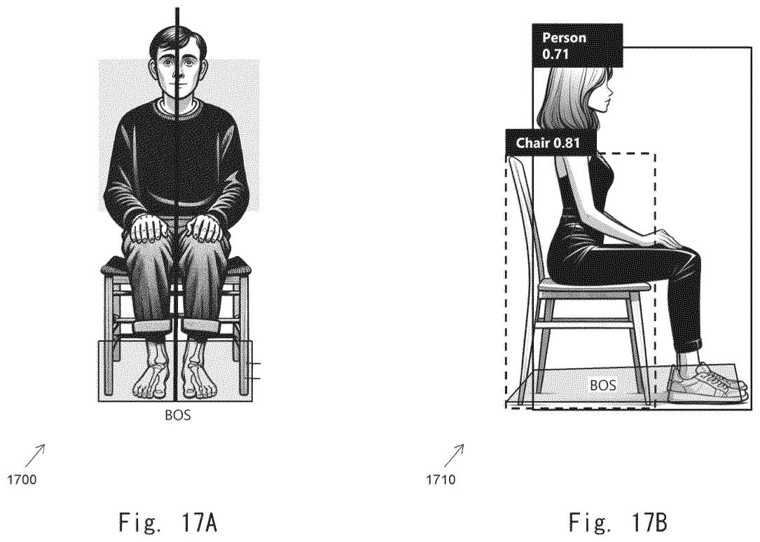

FIGS. 17A and 17B show schematic diagrams 1700, 1710 illustrating BOS of two targets detected from a frontal view image and a side view image when the targets are in a sitting position, respectively, according to an embodiment of the present disclosure. According to this embodiment, both the body of the target and the chair in connection with the body of the target are tracked and their positions and areas contacting the grounds are used for calculating the BOS during sitting and sit to stand and standing.

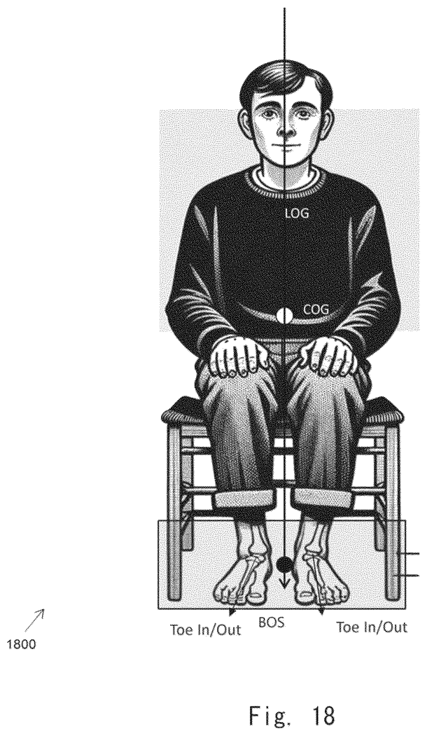

FIG. 18 shows a schematic diagram 1800 illustrating a relationship of BOS, COG and LOG of a target when the target is in a sitting position according to an embodiment of the present disclosure. In this embodiment, the BOS, COG and LOG are detected from the frontal view image of the target. A heel position and a toe position are detected and a lateral rotational angle of the toe relative to the heel can be detected to identify a toe in and toe out position.

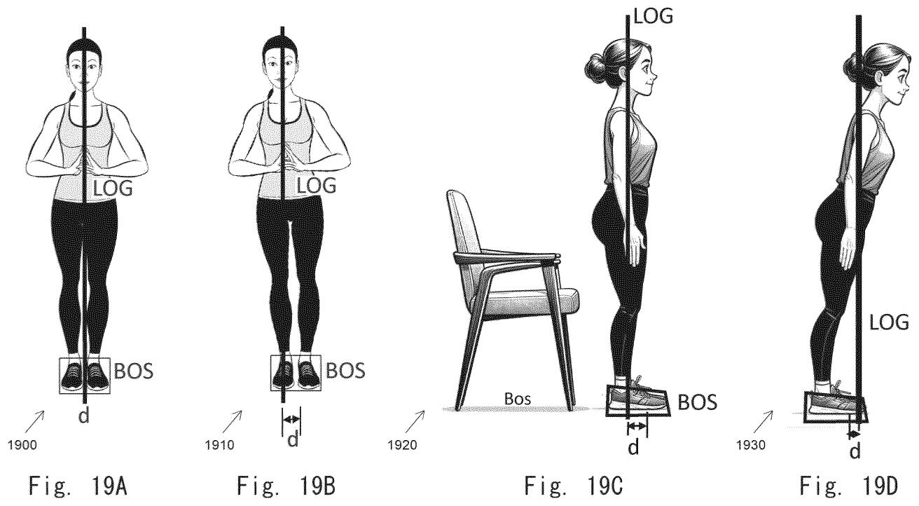

FIGS. 19A and 19B show two schematic diagrams 1900, 1910 illustrating two different standing positions of a target from a frontal view and FIGS. 19C and 19D show another two schematic diagrams 1920, 1930 illustrating two other standing positions of the target from a side view according to an embodiment of the present disclosure, respectively. The COG, BOS of the target in the standing position are detected from the frontal view and side view images, and the LOG of the target can be derived from the COG. The distances (d) between the center of BOS and the LOG are also measured, and compared with a length between two edges of the respective BOS (e.g., BOS/2). As mentioned earlier, LOG must remain within the BOS region to maintain equilibrium and stability. The comparison result may provide information on whether the LOG remain within the BOS region to maintain the biomechanical stability.

According to the present disclosure, the distances between the center of BOS and the LOG detected from each image of the target performing the STS movement will be measured and compared against their a length between two edges of the respective BOS (e.g., BOS/2), and the result of the comparison will be used for calculating a balance and stability index corresponding to the degree of biomechanical stability of the target in performing the STS movement.

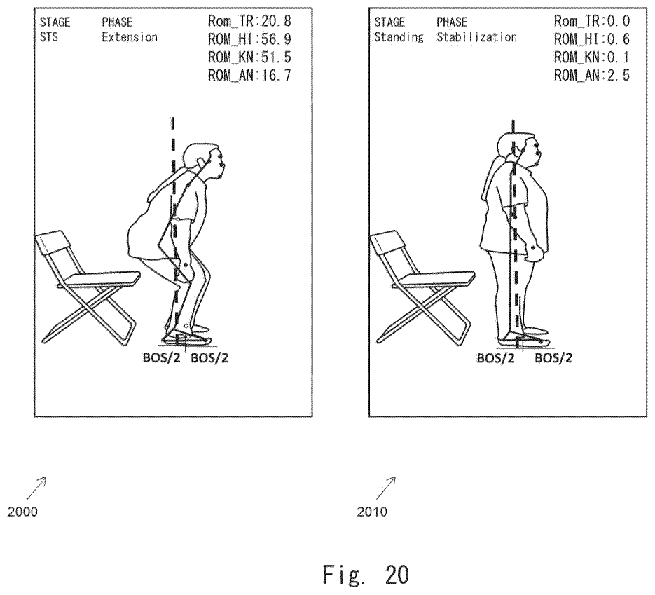

FIG. 20 shows two images 2000, 2010 among a plurality of images of a target captured from a side view during a sit-to-stand movement and the BOS and LOG positions detected from the images according to an embodiment of the present disclosure. Based on the BOS and LOG positions, the distances between the LOGs and the center of the BOSs from images (not shown) of the plurality of images captured throughout the movement from the side view are measured and compared against with a half of distance of BOS to identify the degree of biomechanical stability of the target during the sit-to-stand movement.

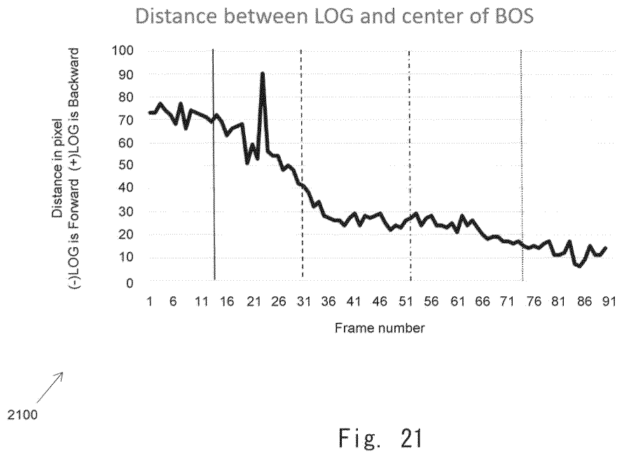

FIG. 21 shows a graph 2100 illustrating exemplary detected distance between LOG and the center of BOS of a target during the STS movement based on side view images according to an embodiment of the present disclosure. Noting that the target can maintain her stability and equilibrium when the distance between the LOG and the center of BOS is less than a half of the distance of BOS (BOS/2, or 121 pixels in this case), the detected distances will be compared with a half of the distance of BOS to identify the biomechanical stability during the movement.

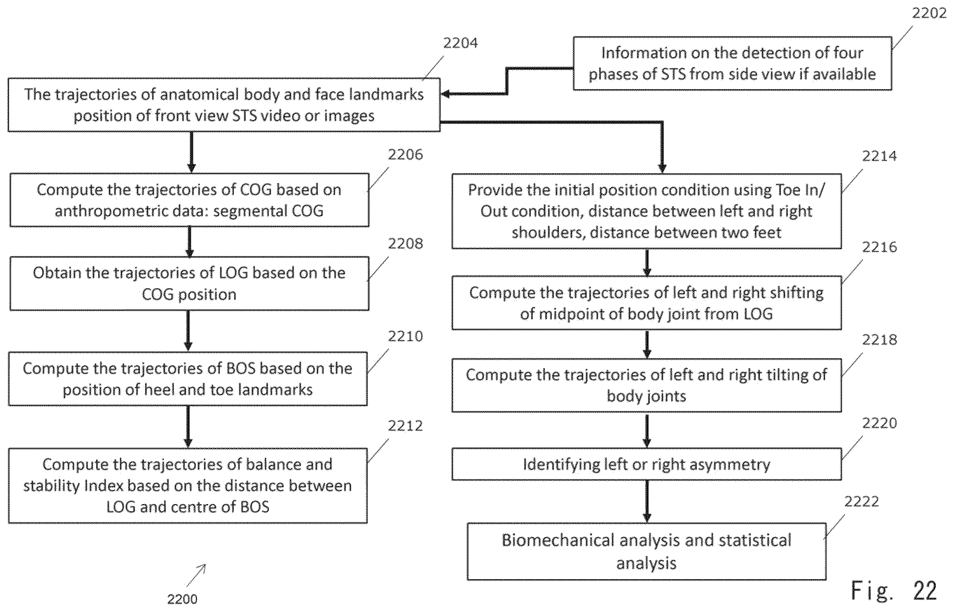

FIG. 22 shows a flow chart 2200 illustrating a method, for example, carried by the dynamic pose analyzer unit 506 in FIG. 5, for processing the landmarks for frontal view according to an embodiment of the present disclosure. In step 2202, the information on the detection of four phases of STS from side view is extracted if available. In step 2204, the trajectories of anatomical body and face landmarks positions of video or images of a target performing an STS movement from the frontal view are identified. In step 2206, a step of computing the trajectories of COG based on anthropometic data (segmental COG) is carried out. In step 2208, a step of obtaining the trajectories of LOG based on the COG position is carried out. In step 2210, a step of computing the trajectories of BOS based on the position of heel and toe landmarks is carried out. In step 2212, a step of trajectories of balance and stability index corresponding to a degree of biomechanical stability based on the distance between LOG and center of BOS is carried out.

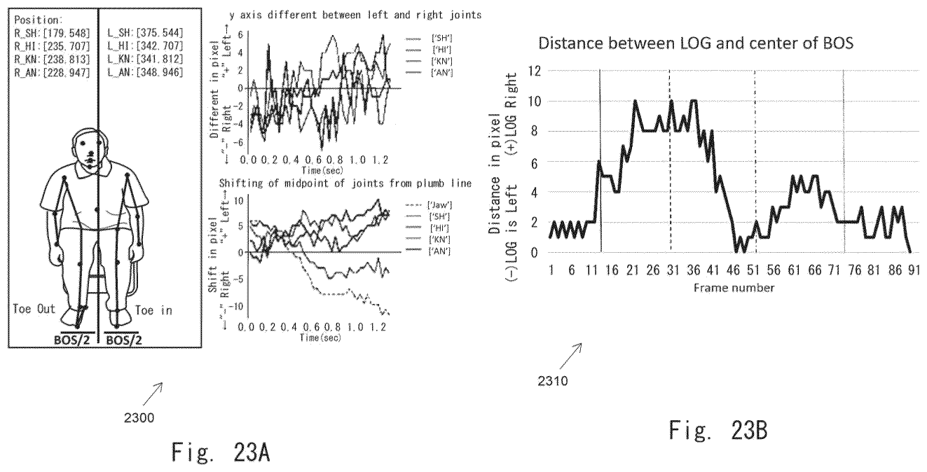

Similarly, based on the frontal view images of the target performing the STS movement, the BOS and LOG of the target during the STS movement are detected from the images. FIG. 23A shows an image 2300 among a plurality of images of a target captured from a frontal view during a sit-to-stand movement, the BOS and LOG positions detected from the image and two graphs illustrating the results of the y-axis difference and shifting of midpoint of joints, according to an embodiment of the present disclosure. Based on the BOS and LOG positions, the distances between the LOGs and the center of the BOSs from the images 2300 and other images (not shown) of the plurality of images captured throughout the movement from the frontal view are measured and compared against with a half of distance of BOS to identify the degree of biomechanical stability of the target during the sit-to-stand movement. FIG. 23B shows a graph 2310 illustrating exemplary detected distance between LOG and the center of BOS of a target during the STS movement based on frontal view images according to an embodiment of the present disclosure. Noting that the target can maintain her stability and equilibrium when the distance between the LOG and the center of BOS is less than a half of the distance of BOS (BOS/2, 103 pixels in this case), the detected distances will be compared with a half of the distance of BOS to identify the biomechanical stability during the movement.

Additionally, in step 2214, the initial position condition using Toe In/out condition, distance between left and right shoulders and distance between two feet are provided. In step 2216, a step of computing the trajectories of left and right shifting of midpoint of body joint from LOG is carried out. In step 2218, a step of computing the trajectories of left and right tilting of body joints is carried out. In step 2220, a step of identifying a left or right asymmetry is carried out. In step 2222, a step of generating a result of biomechanical analysis and statistical analysis (e.g., smoothness, variability) is carried out.

According to the present disclosure, biomechanical analysis and statistical analysis to identify left/right asymmetry may be carried out, for example, in step 2220. FIG. 24 shows a flow chart 2400 illustrating the process of identifying left or right asymmetry according to an embodiment of the present disclosure. In step 2402, the trajectories of anatomical body and face landmarks positions of video or images of a target from the frontal view STS video or images is identified. In step 2404, the trajectories of the distance of left or right joint (e.g., shoulder joint, hip joint, knee joint, ankle joint) to the LOG are identified. Based on the distance, an overall asymmetry index can be calculated through steps 2406 and 2408. In particular, in step 2406, a step of calculating the asymmetry index for trajectories of a joint is carried out using the following equation (2):

$${D}_{right} - {D}_{left} / {D}_{right} + {D}_{left} \qquad equation ( 2 )$$

where D_{right} is the distance of a right joint to the LOG and D_{left} is the distance of a left joint to the LOG.

In step 2408, a step of calculating the overall mean of asymmetry index of the joint in performing the STS movement is carried out. Optionally, in step 2410, the information on the detection of four phases of STS from side view is extracted if available. In step 2412, a step of calculating the mean of asymmetry index for different phases of STS movement is carried out.

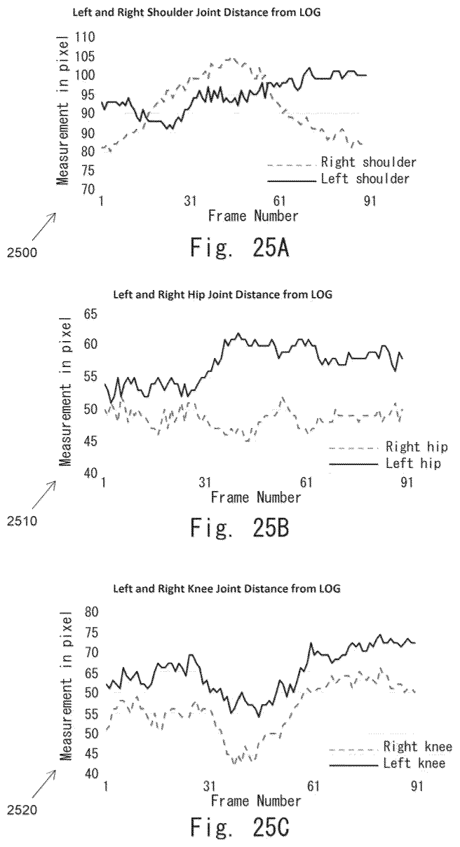

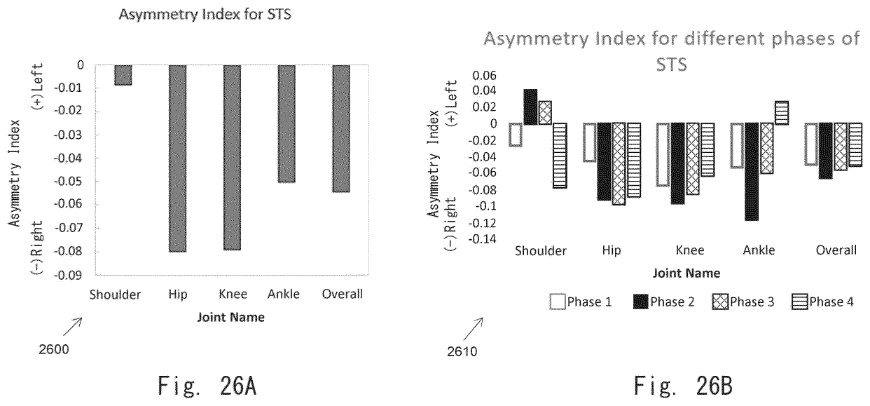

FIGS. 25A to 25C show three graphs 2500, 2510, 2520 illustrating the distances of shoulder joints, hip joints and knee joints to the LOGs of a target measured from frontal view images during an STS movement, respectively, according to an embodiment of the present disclosure. FIG. 26A shows a bar chart 2600 illustrating exemplary asymmetry indices of different body joints for the STS movement derived from frontal STS images according to an embodiment of the present disclosure. FIG. 26B shows another bar chart 2610 exemplary asymmetry indices of different body joints for each phase of the STS movement derived from frontal STS images according to an embodiment of the present disclosure.

These indices provide a measure of asymmetry where negative values indicate right-side dominance and positive values indicate left-side dominance. For all measured joints (shoulders, hips, knees, ankles), the indices are negative, suggesting a tendency towards right-side dominance in the movements. The closer the index is to zero, the more symmetrical the movement between the left and right sides. The further from zero (in either direction), the greater the asymmetry.

From the following results and insights can be derived from the bar charts 2600, 2610:
the asymmetry indices for the major joints (shoulders, hips, knees, and ankles) ranged from −0.08 to −0.03, suggesting a right-side dominance in the movement pattern;
The largest asymmetry was observed in the hip and knee joints, indicating potential imbalances in lower body strength or coordination.
These imbalance and weight bearing are dominance in phase 2 and phase 3.

In addition, in step 2222, biomechanical analysis and statistical analysis relating to the joint may be carried out through other joint movement related parameters derived from the joints-position detected from frontal view images. Such analysis is valuable for understanding joint behavior during movement and identifying areas that may require attention or further analysis, especially in contexts like physical therapy, sports science, or ergonomics.

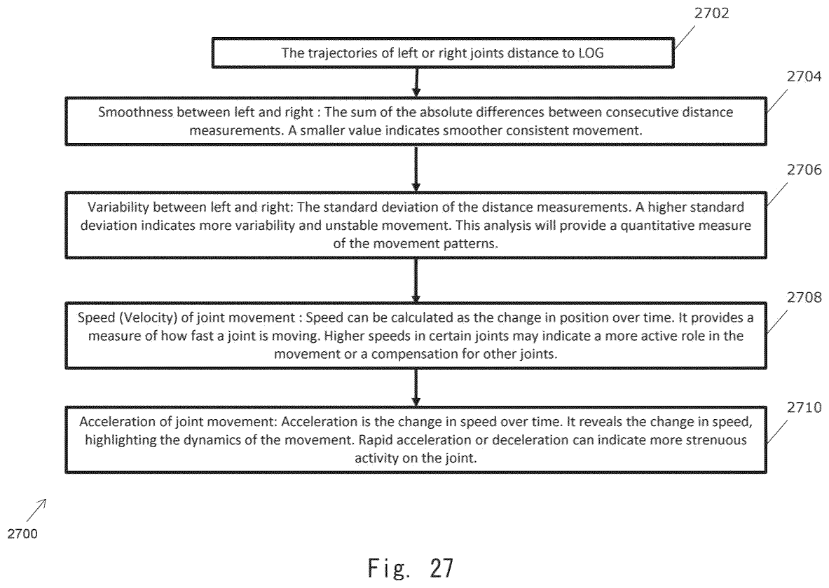

FIG. 27 shows a flow chart 2700 illustrating a process for calculating smoothness between left and right joints, variability between left and right joints, speed (velocity) of joint movement and acceleration of joint movement a target in performing the STS movement according to an embodiment of the present disclosure. In step 2702, trajectories of joint distance to LOG are determined from a series of movement in performing an STS movement from a frontal view video or images. In step 2704, a step of calculating the smoothness between left and right joints is carried out. The smoothness of a left or right joint relates to a sum of the absolute differences between consecutive position of the joint from the series of movement in performing the STS movement from the video or images. It may be equivalent to an average rate of change of the joint in performing the STS movement and can be calculated using equation (1) above. A smaller value of S indicates that there are smaller changes between consecutive measurements, which implies a smoother movement of the joint.

In step 2706, a step of calculating the variability between a left and right joint is carried out. It is the standard deviation of the joints distance to LOG. A higher standard deviation indicates more variability and unstable movement. This analysis will provide a quantitative measure of the movement patterns.

In step 2708, a step of calculating a speed (velocity) of joint movement is carried out. Speed can be calculated as the change in position over time. It provides a measure of how fast a joint is moving. Higher speeds in certain joints may indicate a more active role in the movement or a compensation for other joints. In step 2710, a step of calculating an acceleration of joint movement is carried out. Acceleration is the change in speed over time. It reveals the change in speed, highlighting the dynamics of the movement. Rapid acceleration or deceleration can indicate more strenuous activity on the joint.

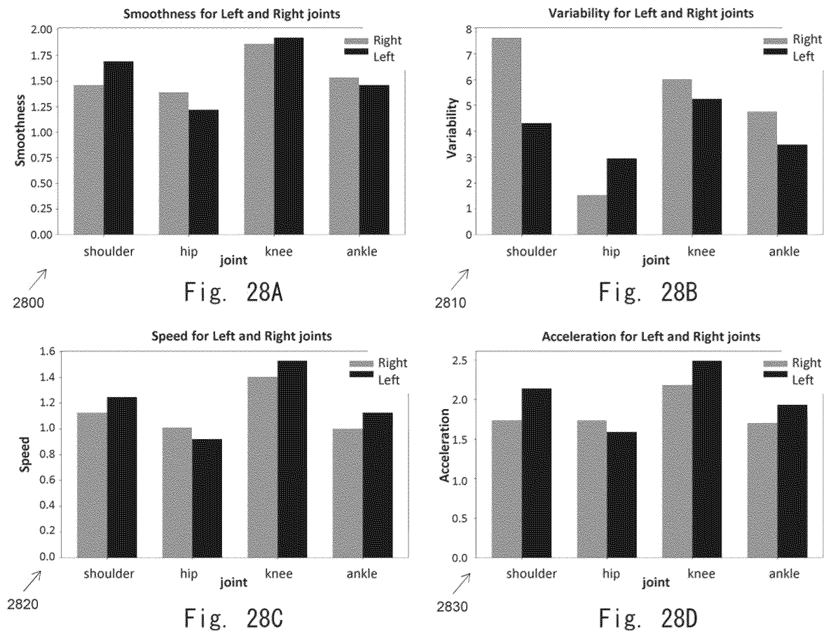

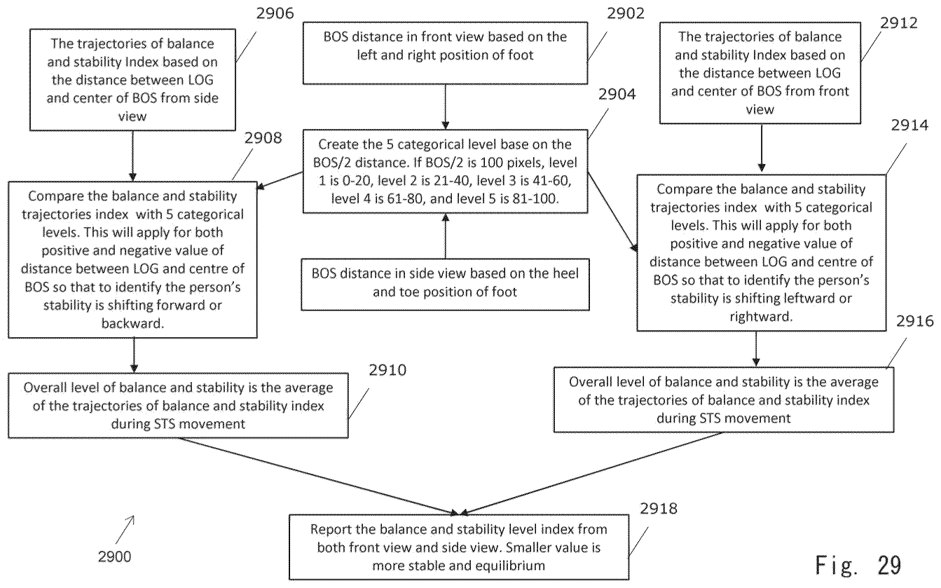

FIGS. 28A to 28D shows four bar graphs 2800, 2810, 2820, 2830, illustrating exemplary results of smoothness, variability, speed and acceleration of left and right joints a target in performing an STS movement according to an embodiment of the present disclosure, respectively. The joints include right ankle joint, right knee joint, right knee joint, right trunk joint, right shoulder joint and right ear joint.

The smoothness bar graph 2800 helps to identify joints with more or less consistent movement throughout the frames. The variability bar graph 2810 helps to identify joints with more stable or unstable movements. Joints with lower bars in the smoothness graph and lower bars in the variability graph are more stable and have smoother movements. Conversely, joints with higher bars in both graphs exhibit more erratic and variable movements. Specific observations about each joint's stability and movement consistency can be made by comparing their respective bars across the two graphs.

The speed bar graph 2820 demonstrates how the average speed for each joint varies between the left and right sides. This visualization can help identify asymmetries in movement speed across the two sides. The acceleration bar graph 2830 shows the differences in the rate of change of speed for each joint between the left and right sides. This can be indicative of how dynamic each joint's movement is, and potentially reveal any imbalances in movement acceleration. These insights can be valuable for understanding the dynamics of movement in different joints and could be useful for applications in biomechanics, physical therapy, or sports science.

From the results, the following insights can be derived:
The analysis reveals distinct patterns of movement and stability across different left and right joints.
The left knee displays the least smoothness, suggesting more erratic movement.
The right shoulder exhibits the highest variability, indicating significant fluctuation in its position across frames.
Hip joints show more consistent and stable movements (high smoothness and low variability).
In conclusion, right shoulder and left knee exhibit more erratic movements (low smoothness and high variability).

Returning to FIG. 6, in step 616, the output from dynamic pose analyzer unit 506 to then be used the processing unit 508 for calculating an overall balance and stability index corresponding to degree of biomechanical stability of the target in performing the STS movement.

FIG. 39 shows a flow chart 2900 illustrating a process of calculating an overall balance and stability index corresponding to degree of biomechanical stability of the target in performing the STS movement according to an embodiment of the present disclosure. It is noted that steps 2906-2910 relate to a processing based on the images from the side view whereas steps 2912-2916 relate to a processing based on the images from the frontal view. In step 2902, a BOS distance in front view is calculated based on the left and right position of foot. In step 2904, five categorical levels are created based the half of the BOS length (BOS/2). For example, if BOS/2 is 100 pixels, level 1 corresponds to 0-20 pixels, level 2 corresponds to 21-40 pixels, level 3 corresponds to 41-60 pixels, level 4 corresponds to 61-80 pixels and level 5 corresponds to 81-100 pixels.

In step 2906, the trajectories of balance and stability index are detected based on the distance between the LOG and the center of BOS from side view. In step 2908, the balance and stability trajectories index is compared with the 5 categorical levels to determine which level the index falls into. This will apply for both positive and negative value of distance between LOG and center of BOS so that to identify the person's stability is shifting forward or backward. In step 2910, the overall level of balance and stability, which is the average of the trajectories of balance and stability index during STS movement, is calculated.

In step 2912, the trajectories of balance and stability index are detected based on the distance between the LOG and the center of BOS from frontal view. In step 2914, the balance and stability trajectories index is compared with the 5 categorical levels to determine which level the index falls into. This will apply for both positive and negative value of distance between LOG and center of BOS so that to identify the person's stability is shifting leftward or rightward. In step 2916, the overall level of balance and stability, which is the average of the trajectories of balance and stability index during STS movement, is calculated. In step 2918, the balance and stability level index from both frontal view and side view is reported. A smaller value of the index indicates a greater degree of biomechanical stability, meaning that the target is more stable and equilibrium in performing the STS movement.

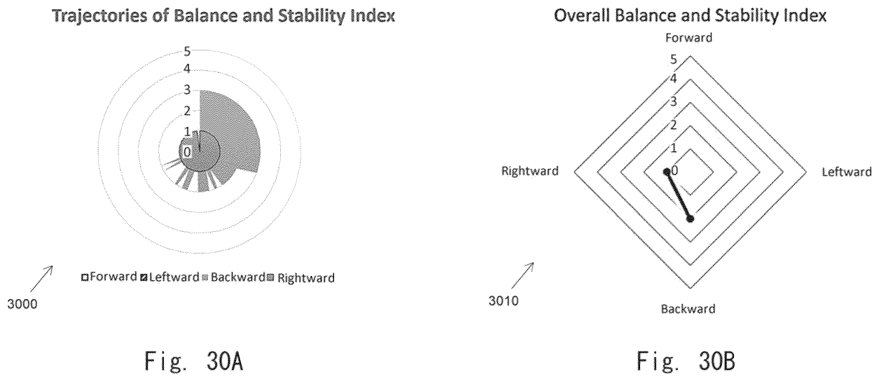

FIG. 30A shows a radar chart 3000 illustrating trajectories of balance and stability index detected from side and frontal view images, for example in steps 2906, 2912, according to an embodiment of the present disclosure. FIG. 30B shows another radar chart 3010 illustrating the overall balance and stability index output from step 2918 according to an embodiment of the present disclosure.

The following results and insights can be derived from the charts 3000, 3010:
from the frontal view perspective, the line of gravity falls to the right side of the base of support during a sit-to-stand movement, the person's weight is shifting more to the right side. This could be due to weakness, imbalance, or a strategy to compensate for some other issue
from the side view perspective, the line of gravity is behind the center of the base of support during the sit-to-stand movement, it suggests that the individual is leaning backwards. This could occur if a person leans back to use the momentum to stand up, possibly due to weakness in the legs or trunk, or to avoid pain or discomfort in the knees.

Returning to FIG. 6, in step 618, the overall balance and stability index will then be used by a large language model unit 510 to generate physiotherapy insights. In step 620, the sit-to-stand assessment including biomechanics analysis and physiotherapy insights is reported. In step 622, a sit-to-stand assessment summary is reported for clinician's review, for example, displaying in the user interface 512.

The goal in a sit-to-stand movement is usually to keep the line of gravity within the base of support to maintain balance and ensure a smooth transition to standing. A proper sit-to-stand movement typically involves shifting the center of mass forward and over the feet, so that the line of gravity remains within the base of support for stability. Deviations from this can indicate a need for physical therapy or alterations in technique to improve safety and efficiency.

Below is an example STS assessment summary for Clinician's Review:

### Right ROM Trajectories

### Smoothness:

Shoulders exhibited the smoothest movement, indicating controlled, consistent motion. This smoothness in shoulder movement is positive for upper body stability.
Hip and knee joints showed less smoothness, suggesting more dynamic or possibly less controlled movements. This could indicate challenges in maintaining stability during activities involving significant lower limb movement.

### Variability:

High variability in hip and knee joints suggests a broad range of motion. This can be advantageous for flexibility but may also point to potential instability in weight-bearing and balance tasks.
Low variability in the shoulder joint indicates consistency in movement, beneficial for tasks requiring upper body steadiness.

### Front Midpoint Shifting

### Asymmetry Index:

The values obtained indicate the degree of symmetry in movement between the right and left sides. Asymmetries, especially in the hip and knee, can signify potential issues with balance and weight distribution.
Asymmetries in lower joints (hip, knee) are particularly crucial as they directly impact gait and weight-bearing capabilities.

### Similarity:

High correlation coefficients would suggest synchronized movements between the left and right sides, which is essential for balanced weight bearing and coordinated movement.
Any deviations might indicate a disparity in muscle strength or joint function between the two sides, which could affect balance and necessitate further investigation or intervention.

### Medical Implications

### Balance and Weight Bearing:

The observed asymmetries and variability in the hip and knee joints could affect balance, particularly during dynamic movements. This could be a concern for activities requiring equal weight distribution and coordination, such as walking or climbing stairs.
Shoulder stability, as indicated by low variability and high smoothness, is a positive sign for upper body balance but should be considered in conjunction with lower limb stability.

### Therapeutic Considerations:

For patients with imbalance or gait issues, targeted exercises to improve hip and knee stability and symmetry might be beneficial.
Monitoring and possibly improving the smoothness of movements in the hip and knee joints could enhance overall stability and reduce the risk of falls or injuries.

### Further Assessment:

These findings should be corroborated with a physical examination and possibly further functional assessments to understand the patient's overall balance and weight-bearing capabilities in real-world scenarios.
This analysis provides a basis for understanding potential biomechanical issues but should be integrated with a full clinical assessment for a comprehensive understanding of the patient's needs.

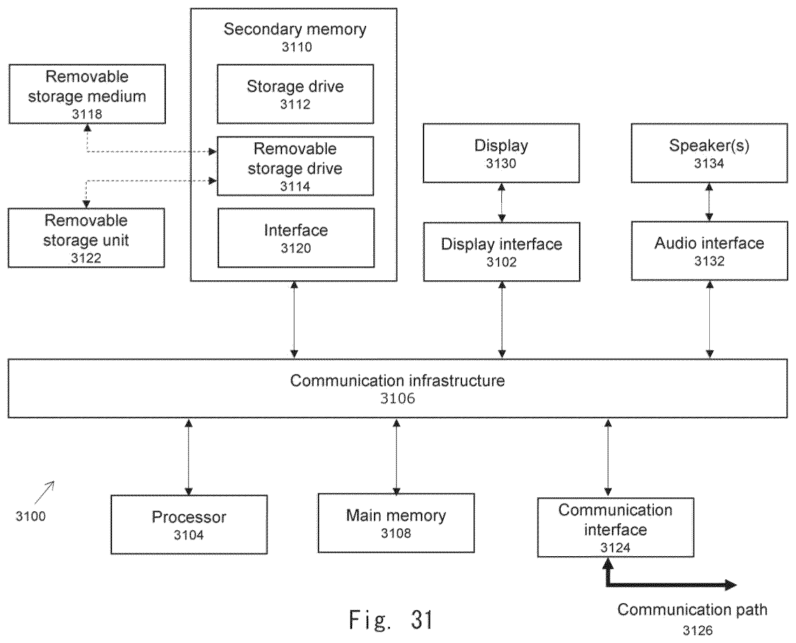

FIG. 31 depicts an exemplary computing device 3100, hereinafter interchangeably referred to as a computer system 3100, where one or more such computing devices 3100 may be used to execute the method of FIG. 4. The exemplary computing device 3100 can be used to implement the apparatus 300 shown in FIG. 3. The following description of the computing device 3100 is provided by way of example only and is not intended to be limiting.

As shown in FIG. 31, the example computing device 3100 includes a processor 3104 for executing software routines. Although a single processor is shown for the sake of clarity, the computing device 3100 may also include a multi-processor system. The processor 3104 is connected to a communication infrastructure 3106 for communication with other components of the computing device 3100. The communication infrastructure 3106 may include, for example, a communications bus, cross-bar, or network.

The computing device 3100 further includes a main memory 3108, such as a random access memory (RAM), and a secondary memory 3110. The secondary memory 3110 may include, for example, a storage drive 3112, which may be a hard disk drive, a solid state drive or a hybrid drive and/or a removable storage drive 3114, which may include a magnetic tape drive, an optical disk drive, a solid state storage drive (such as a USB flash drive, a flash memory device, a solid state drive or a memory card), or the like. The removable storage drive 3114 reads from and/or writes to a removable storage medium 3118 in a well-known manner. The removable storage medium 3118 may include magnetic tape, optical disk, non-volatile memory storage medium, or the like, which is read by and written to by removable storage drive 3114. As will be appreciated by persons skilled in the relevant art(s), the removable storage medium 3118 includes a computer readable storage medium having stored therein computer executable program code instructions and/or data.

In an alternative implementation, the secondary memory 3110 may additionally or alternatively include other similar means for allowing computer programs or other instructions to be loaded into the computing device 3100. Such means can include, for example, a removable storage unit 3122 and an interface 2310. Examples of a removable storage unit 3122 and interface 2420 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 3122 and interfaces 2420 which allow software and data to be transferred from the removable storage unit 3122 to the computer system 3100.

The computing device 3100 also includes at least one communication interface 3124. The communication interface 3124 allows software and data to be transferred between computing device 3100 and external devices via a communication path 3126. In various embodiments of the inventions, the communication interface 3124 permits data to be transferred between the computing device 3100 and a data communication network, such as a public data or private data communication network. The communication interface 3124 may be used to exchange data between different computing devices 600 which such computing devices 3100 form part an interconnected computer network. Examples of a communication interface 3124 can include a modem, a network interface (such as an Ethernet card), a communication port (such as a serial, parallel, printer, GPIB, IEEE 1394, RJ45, USB), an antenna with associated circuitry and the like. The communication interface 3124 may be wired or may be wireless. Software and data transferred via the communication interface 3124 are in the form of signals which can be electronic, electromagnetic, optical or other signals capable of being received by communication interface 3124. These signals are provided to the communication interface via the communication path 3126.

As shown in FIG. 31, the computing device 3100 further includes a display interface 3102 which performs operations for rendering images to an associated display 3130 and an audio interface 3132 for performing operations for playing audio content via associated speaker(s) 3131.

As used herein, the term “computer program product” may refer, in part, to removable storage medium 3118, removable storage unit 3122, a hard disk installed in storage drive 3112, or a carrier wave carrying software over communication path 3126 (wireless link or cable) to communication interface 3124. Computer readable storage media refers to any non-transitory, non-volatile tangible storage medium that provides recorded instructions and/or data to the computing device 3100 for execution and/or processing. Examples of such storage media include magnetic tape, CD-ROM, DVD, Blu-ray Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a PCMCIA card and the like, whether or not such devices are internal or external of the computing device 3100. Examples of transitory or non-tangible computer readable transmission media that may also participate in the provision of software, application programs, instructions and/or data to the computing device 3100 include radio or infra-red transmission channels as well as a network connection to another computer or networked device, and the Internet or Intranets including e-mail transmissions and information recorded on Websites and the like.

The computer programs (also called computer program code) are stored in main memory 3108 and/or secondary memory 3110. Computer programs can also be received via the communication interface 3124. Such computer programs, when executed, enable the computing device 3100 to perform one or more features of embodiments discussed herein. In various embodiments, the computer programs, when executed, enable the processor 3104 to perform features of the above-described embodiments. Accordingly, such computer programs represent controllers of the computer system 3100.

Software may be stored in a computer program product and loaded into the computing device 3100 using the removable storage drive 3114, the storage drive 3112, or the interface 3120. The computer program product may be a non-transitory computer readable medium. Alternatively, the computer program product may be downloaded to the computer system 3100 over the communications path 3126. The software, when executed by the processor 3104, causes the computing device 3100 to perform the necessary operations to execute the method as shown in FIG. 4.

It is to be understood that the embodiment of FIG. 31 is presented merely by way of example to explain the operation and structure of the apparatus 300. Therefore, in some embodiments one or more features of the computing device 3100 may be omitted. Also, in some embodiments, one or more features of the computing device 3100 may be combined together. Additionally, in some embodiments, one or more features of the computing device 3100 may be split into one or more component parts.

Thus, it can be seen that a method, an apparatus and a system for determining a biomechanical stability of a target in performing a movement from a sitting position to a standing position has been provided. Such method, apparatus and system enhance objectivity and precision in healthcare, an automated system is crucial for conducting sit-to-stand evaluations, providing more valuable biomechanical and physiotherapy insights in both clinical and home settings beyond subjective assessments.

It will be appreciated by a person skilled in the art that numerous variations and/or modifications may be made to the present disclosure as shown in the specific embodiments without departing from the spirit or scope of the disclosure as broadly described. The present embodiments are, therefore, to be considered in all respects to be illustrative and not restrictive.

The whole or part of the exemplary embodiments disclosed above can be described as, but not limited to, the following supplementary notes.

### Supplementary Note 1

A method for determining a biomechanical stability of a target in performing a movement from a sitting position to a standing position comprising:
identifying, from each of a plurality of images, (i) an area on a ground which the target is in contact with and (i) a line of gravity of the target projecting from a center of gravity of the target and perpendicular to the ground, wherein the each of the plurality of images showing one of a series of movements of the target moving from the sitting position to the standing position; and
calculating a degree of biomechanical stability of the target in performing the movement from the sitting position to the standing position based on the areas and the lines of gravity of the target identified from the plurality of images.

### Supplementary Note 2

The method of supplementary note 1, further comprising:
measuring, from the each of the plurality of images, a shortest distance from the center of the area to a point on the line of gravity, wherein the degree of biomechanical stability is calculated based on the shortest distance.

### Supplementary Note 3

The method of supplementary note 2, further comprising:
comparing the shortest distance with a half of a length between two edges of the area on the ground, wherein the degree of biomechanical stability is calculated based on a result of the comparison.

### Supplementary Note 4

The method of any one of supplementary notes 1-3, further comprising:
identifying, from the each of the plurality of images, a position of a center of gravity of each of body segments of the target at a pre-configured length, width and/or height of the each of the body segments; and
determining the center of gravity of the target based on the position of the center of gravity of the body segment and a pre-configured weight percentage of the body segments making up a total weight of the target.

### Supplementary Note 5

The method of any one of supplementary notes 1-4, further comprising:
detecting, from the each of the plurality of images, positions of a toe, a heel and/or a foot of the target, wherein the area on the ground which the target is in contact with are identified based on the positions of the toe, the heel and/or the foot of the target.

### Supplementary Note 6

The method of any one of supplementary notes 1-5, wherein there are four stages of movements performed by the target to complete the movement from the sitting position to the standing position, further comprising:
calculating, from the each of the plurality of images, one of (i) a first displacement of a position of a hip of the target along a direction parallel to the line of gravity, (ii) a second displacement of a position of a shoulder of the target along the direction parallel to the line of gravity, (iii) a first relative angle between two first body segments adjacent to the position of the hip; and (iv) a second relative angle between two second body segments adjacent to a position of a knee of the target; (v) a third relative angle between two third body segments adjacent to a position of an ankle of the target;
categorizing the series of movements under one of four stages of movements performed by the target to complete the movement from the sitting position to the standing position based on the one of (i) the first displacement of the position of the hip, (ii) the second displacement of the position of the shoulder, (iii) the first relative angle between the two first body segments adjacent to the position of the hip, (iv) the second relative angle between the two second body segments adjacent to the position of the knee of the target and (v) the third relative angle between the two third body segments adjacent to the position of the ankle of the target.

### Supplementary Note 7

The method of any one of supplementary notes 1-6, further comprising:
measuring, from the each of the plurality of images, a relative angle formed between two fourth body segments adjacent to a position of a joint, wherein the joint is one of a hip, a knee, a hip, a trunk, a shoulder and an ear of the target;
calculating a change in the relative angles between the two fourth body segments around the position of joint measured across the plurality of images, wherein a smaller change or rate of the change in the relative angles indicates a smoothness of a movement of the joint in the series of movements.

### Supplementary Note 8

The method of supplementary note 7, further comprising:
calculating a standard deviation of the relative angles between the two body segments around the position of the joint measured across the plurality of images, wherein a smaller standard deviation indicates a lower variability or greater stability of the movement of the joint in the series of movements.

### Supplementary Note 9

The method of supplementary note 7 or 8, further comprising:
calculating a speed and/or an acceleration of movements of the joint across the plurality of images; wherein the speed and/or the acceleration indicating an activity of the joint in the series of movements.

### Supplementary Note 10

The method of any one of supplementary notes 1-6, further comprising:
measuring, from the each of the plurality of images, shortest distances from positions of a left joint and a right joint of a joint to a point on the line of gravity, wherein the joint is one of a hip, a knee, an ankle, a trunk, a shoulder and an ear of the target; and
calculating a degree of asymmetry between the left joint and the right joint in the series of movements based on the distances measured from the each of the plurality of images.

### Supplementary Note 11

The method of any one of supplementary notes 1-10, wherein the plurality of images comprises a first plurality of images showing the series of movements of the target moving from the sitting position to the standing position from a side view, and the step of calculating the degree of biomechanical stability comprises calculating a first degree of biomechanical stability of the target along a forward and/or backward direction based on the area and the lines of gravity of the target identified from the first plurality of images.

### Supplementary Note 12

The method of any one of supplementary notes 1-11, wherein the plurality of images comprises a second plurality of images showing the series of movements of the target moving from the sitting position to the standing position from a frontal view, and the step of calculating the degree of biomechanical stability comprising calculating a second degree of biomechanical stability of the target along a right and/or left direction based on the area and the lines of gravity of the target identified from the second plurality of images.

### Supplementary Note 13

An apparatus for determining a biomechanical stability of a target in performing a movement from a sitting position to a standing position comprising:
at least one processor; and
at least one memory including computer program code;
the at least one memory and the computer program code configured to, with at least one processor, cause the apparatus at least to:
   identify, from each of a plurality of images, (i) an area on a ground which the target is in contact with and (i) a line of gravity of the target projecting from a center of gravity of the target and perpendicular to the ground, wherein the each of the plurality of images showing one of a series of movements of the target moving from the sitting position to the standing position; and
   calculate a degree of biomechanical stability of the target in performing the movement from the sitting position to the standing position based on the areas and the lines of gravity of the target identified from the plurality of images.

### Supplementary Note 14

The apparatus of supplementary note 13, the at least one memory and the computer program code configured to, with at least one processor, cause the apparatus at least to further:
measure, from the each of the plurality of images, a shortest distance from the center of the area to a point on the line of gravity, wherein the degree of biomechanical stability is calculated based on the shortest distance.

### Supplementary Note 15

The apparatus of supplementary note 14, the at least one memory and the computer program code configured to, with at least one processor, cause the apparatus at least to further:
compare the shortest distance with a half of a length between two edges of the area on the ground, wherein the degree of biomechanical stability is calculated based on a result of the comparison.

### Supplementary Note 16

The apparatus of any one of supplementary notes 13-15, the at least one memory and the computer program code configured to, with at least one processor, cause the apparatus at least to further:
identify, from the each of the plurality of images, a position of a center of gravity of each of body segments of the target at a pre-configured length, width and/or height of the each of the body segments; and
determine the center of gravity of the target based on the position of the center of gravity of the body segment and a pre-configured weight percentage of the body segments making up a total weight of the target.

### Supplementary Note 17

The apparatus of any one of supplementary notes 13-16, the at least one memory and the computer program code configured to, with at least one processor, cause the apparatus at least to further:
detect, from the each of the plurality of images, positions of a toe, a heel and/or a foot of the target, wherein the area on the ground which the target is in contact with are identified based on the positions of the toe, the heel and/or the foot of the target.

### Supplementary Note 18

The apparatus of any one of supplementary notes 13-17, wherein there are four stages of movements performed by the target to complete the movement from the sitting position to the standing position, the at least one memory and the computer program code configured to, with at least one processor, cause the apparatus at least to further:
calculate, from the each of the plurality of images, one of (i) a first displacement of a position of a hip of the target along a direction parallel to the line of gravity, (ii) a second displacement of a position of a shoulder of the target along the direction parallel to the line of gravity, (iii) a first relative angle between two first body segments adjacent to the position of the hip; and (iv) a second relative angle between two second body segments adjacent to a position of a knee of the target; and (v) a third relative angle between two third body segments adjacent to a position of an ankle of the target;
categorize the series of movements under one of four stages of movements performed by the target to complete the movement from the sitting position to the standing position based on the one of (i) the first displacement of the position of the hip, (ii) the second displacement of the position of the shoulder, (iii) the first relative angle between the two first body segments adjacent to the position of the hip, (iv) the second relative angle between the two second body segments adjacent to the position of the knee of the target and (v) the third relative angle between the two third body segments adjacent to the position of the ankle of the target.

### Supplementary Note 19

The apparatus of any one of supplementary notes 13-18, the at least one memory and the computer program code configured to, with at least one processor, cause the apparatus at least to further:
Measure, from the each of the plurality of images, a relative angle formed between two fourth body segments adjacent to a position of a joint, wherein the joint is one of a hip, a knee, an ankle, a trunk, a shoulder and an ear of the target;
calculate a change in the relative angles between the two fourth body segments around the position of joint measured across the plurality of images, wherein a smaller change or rate of the change in the relative angles indicates a smoothness of a movement of the joint in the series of movements.

### Supplementary Note 20

The apparatus of supplementary note 19, the at least one memory and the computer program code configured to, with at least one processor, cause the apparatus at least to further:
calculate a standard deviation of the relative angles between the two body segments around the position of the joint measured across the plurality of images, wherein a smaller standard deviation indicates a lower variability or greater stability of the movement of the joint in the series of movements.

### Supplementary Note 21

The apparatus of supplementary note 19 or 20, the at least one memory and the computer program code configured to, with at least one processor, cause the apparatus at least to further:
calculate a speed and/or an acceleration of movements of the joint across the plurality of images; wherein the speed and/or the acceleration indicating an activity of the joint in the series of movements.

### Supplementary Note 22

The apparatus of any one of supplementary notes 13-18, the at least one memory and the computer program code configured to, with at least one processor, cause the apparatus at least to further:
measure, from the each of the plurality of images, shortest distances from positions of a left joint and a right joint of a joint to a point on the line of gravity, wherein the joint is one of a hip, a knee, an ankle, a trunk, a shoulder and an ear of the target; and
calculate a degree of asymmetry between the left joint and the right joint in the series of movements based on the distances measured from the each of the plurality of images.

### Supplementary Note 23

The apparatus of any one of supplementary notes 13-22, wherein the plurality of images comprises a first plurality of images showing the series of movements of the target moving from the sitting position to the standing position from a side view, the at least one memory and the computer program code configured to, with at least one processor, cause the apparatus at least to further:
calculate a first degree of biomechanical stability of the target along a forward and/or backward direction based on the area and the lines of gravity of the target identified from the first plurality of images.

### Supplementary Note 24

The apparatus of any one of supplementary notes 13-23, wherein the plurality of images comprises a second plurality of images showing the series of movements of the target moving from the sitting position to the standing position from a frontal view, and the at least one memory and the computer program code configured to, with at least one processor, cause the apparatus at least to further:
calculate a second degree of biomechanical stability of the target along a right and/or left direction based on the area and the lines of gravity of the target identified from the second plurality of images.

### Supplementary Note 25

A system for determining a biomechanical stability of a target in performing a movement from a sitting position to a standing position comprising an apparatus according to any one of supplementary notes 13-24 and an image capturing apparatus for capturing the plurality of images and detecting the series of movements of the target from the plurality of images.

A (The) program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example, to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

1. A method for determining a biomechanical stability of a target in performing a movement from a sitting position to a standing position comprising:
identifying, from each of a plurality of images, (i) an area on a ground which the target is in contact with and (i) a line of gravity of the target projecting from a center of gravity of the target and perpendicular to the ground, wherein the each of the plurality of images showing one of a series of movements of the target moving from the sitting position to the standing position; and
calculating a degree of biomechanical stability of the target in performing the movement from the sitting position to the standing position based on the areas and the lines of gravity of the target identified from the plurality of images.

2. The method of claim 1, further comprising:
measuring, from the each of the plurality of images, a shortest distance from the center of the area to a point on the line of gravity, wherein the degree of biomechanical stability is calculated based on the shortest distance.

3. The method of claim 2, further comprising:
comparing the shortest distance with a half of a length between two edges of the area on the ground, wherein the degree of biomechanical stability is calculated based on a result of the comparison.

4. The method of claim 1, further comprising:
identifying, from the each of the plurality of images, a position of a center of gravity of each of body segments of the target at a pre-configured length, width and/or height of the each of the body segments; and
determining the center of gravity of the target based on the position of the center of gravity of the body segment and a pre-configured weight percentage of the body segments making up a total weight of the target.

5. The method of claim 1, further comprising:
detecting, from the each of the plurality of images, positions of a toe, a heel and/or a foot of the target, wherein the area on the ground which the target is in contact with are identified based on the positions of the toe, the heel and/or the foot of the target.

6. The method of claim 1, wherein there are four stages of movements performed by the target to complete the movement from the sitting position to the standing position, further comprising:
calculating, from the each of the plurality of images, one of (i) a first displacement of a position of a hip of the target along a direction parallel to the line of gravity, (ii) a second displacement of a position of a shoulder of the target along the direction parallel to the line of gravity, (iii) a first relative angle between two first body segments adjacent to the position of the hip; and (iv) a second relative angle between two second body segments adjacent to a position of a knee of the target; (v) a third relative angle between two third body segments adjacent to a position of an ankle of the target;
categorizing the series of movements under one of four stages of movements performed by the target to complete the movement from the sitting position to the standing position based on the one of (i) the first displacement of the position of the hip, (ii) the second displacement of the position of the shoulder, (iii) the first relative angle between the two first body segments adjacent to the position of the hip, (iv) the second relative angle between the two second body segments adjacent to the position of the knee of the target and (v) the third relative angle between the two third body segments adjacent to the position of the ankle of the target.

7. The method of claim 1, further comprising:
measuring, from the each of the plurality of images, a relative angle formed between two fourth body segments adjacent to a position of a joint, wherein the joint is one of a hip, a knee, a hip, a trunk, a shoulder and an ear of the target;
calculating a change in the relative angles between the two fourth body segments around the position of joint measured across the plurality of images, wherein a smaller change or rate of the change in the relative angles indicates a smoothness of a movement of the joint in the series of movements.

8. The method of claim 7, further comprising:
calculating a standard deviation of the relative angles between the two body segments around the position of the joint measured across the plurality of images, wherein a smaller standard deviation indicates a lower variability or greater stability of the movement of the joint in the series of movements.

9. The method of claim 7, further comprising:
calculating a speed and/or an acceleration of movements of the joint across the plurality of images; wherein the speed and/or the acceleration indicating an activity of the joint in the series of movements.

10. The method of claim 1, further comprising:
measuring, from the each of the plurality of images, shortest distances from positions of a left joint and a right joint of a joint to a point on the line of gravity, wherein the joint is one of a hip, a knee, an ankle, a trunk, a shoulder and an ear of the target; and
calculating a degree of asymmetry between the left joint and the right joint in the series of movements based on the distances measured from the each of the plurality of images.

11. The method of claim 1, wherein the plurality of images comprises a first plurality of images showing the series of movements of the target moving from the sitting position to the standing position from a side view, and the step of calculating the degree of biomechanical stability comprises calculating a first degree of biomechanical stability of the target along a forward and/or backward direction based on the area and the lines of gravity of the target identified from the first plurality of images.

12. The method of claim 1, wherein the plurality of images comprises a second plurality of images showing the series of movements of the target moving from the sitting position to the standing position from a frontal view, and the step of calculating the degree of biomechanical stability comprising calculating a second degree of biomechanical stability of the target along a right and/or left direction based on the area and the lines of gravity of the target identified from the second plurality of images.

13. An apparatus for determining a biomechanical stability of a target in performing a movement from a sitting position to a standing position comprising:
at least one processor; and
at least one memory including computer program code;
the at least one memory and the computer program code configured to, with at least one processor, cause the apparatus at least to:
identify, from each of a plurality of images, (i) an area on a ground which the target is in contact with and (i) a line of gravity of the target projecting from a center of gravity of the target and perpendicular to the ground, wherein the each of the plurality of images showing one of a series of movements of the target moving from the sitting position to the standing position; and
calculate a degree of biomechanical stability of the target in performing the movement from the sitting position to the standing position based on the areas and the lines of gravity of the target identified from the plurality of images.

14. The apparatus of claim 13, the at least one memory and the computer program code configured to, with at least one processor, cause the apparatus at least to further:
measure, from the each of the plurality of images, a shortest distance from the center of the area to a point on the line of gravity, wherein the degree of biomechanical stability is calculated based on the shortest distance.

15. The apparatus of claim 14, the at least one memory and the computer program code configured to, with at least one processor, cause the apparatus at least to further:
compare the shortest distance with a half of a length between two edges of the area on the ground, wherein the degree of biomechanical stability is calculated based on a result of the comparison.

16. The apparatus of claim 13, the at least one memory and the computer program code configured to, with at least one processor, cause the apparatus at least to further:
identify, from the each of the plurality of images, a position of a center of gravity of each of body segments of the target at a pre-configured length, width and/or height of the each of the body segments; and
determine the center of gravity of the target based on the position of the center of gravity of the body segment and a pre-configured weight percentage of the body segments making up a total weight of the target.

17. The apparatus of claim 13, the at least one memory and the computer program code configured to, with at least one processor, cause the apparatus at least to further:
detect, from the each of the plurality of images, positions of a toe, a heel and/or a foot of the target, wherein the area on the ground which the target is in contact with are identified based on the positions of the toe, the heel and/or the foot of the target.

18. The apparatus of claim 13, wherein there are four stages of movements performed by the target to complete the movement from the sitting position to the standing position, the at least one memory and the computer program code configured to, with at least one processor, cause the apparatus at least to further:
calculate, from the each of the plurality of images, one of (i) a first displacement of a position of a hip of the target along a direction parallel to the line of gravity, (ii) a second displacement of a position of a shoulder of the target along the direction parallel to the line of gravity, (iii) a first relative angle between two first body segments adjacent to the position of the hip; and (iv) a second relative angle between two second body segments adjacent to a position of a knee of the target; and (v) a third relative angle between two third body segments adjacent to a position of an ankle of the target;
categorize the series of movements under one of four stages of movements performed by the target to complete the movement from the sitting position to the standing position based on the one of (i) the first displacement of the position of the hip, (ii) the second displacement of the position of the shoulder, (iii) the first relative angle between the two first body segments adjacent to the position of the hip, (iv) the second relative angle between the two second body segments adjacent to the position of the knee of the target and (v) the third relative angle between the two third body segments adjacent to the position of the ankle of the target.

19. The apparatus of claim 13, the at least one memory and the computer program code configured to, with at least one processor, cause the apparatus at least to further:
Measure, from the each of the plurality of images, a relative angle formed between two fourth body segments adjacent to a position of a joint, wherein the joint is one of a hip, a knee, an ankle, a trunk, a shoulder and an ear of the target;
calculate a change in the relative angles between the two fourth body segments around the position of joint measured across the plurality of images, wherein a smaller change or rate of the change in the relative angles indicates a smoothness of a movement of the joint in the series of movements.

20. A system for determining a biomechanical stability of a target in performing a movement from a sitting position to a standing position comprising an apparatus according to claim 13 and an image capturing apparatus for capturing the plurality of images and detecting the series of movements of the target from the plurality of images.
